(19) United States
(12) Patent Application Publication
(10) Pub. No.: US 2018/0251820 A1
(43) Pub. Date: Sep. 6, 2018

(54) COMPOSITIONS AND METHODS FOR DIAGNOSING COLON DISORDERS

(71) Applicants: George Mason Research Foundation, Inc., Fairfax, VA (US); RUSH UNIVERSITY, CHICAGO, IL (US)

(72) Inventors: Patrick M. Gillevet, Oakton, VA (US); Ali Keshavarzian, Evanston, IL (US)

(21) Appl. No.: 15/983,906

(22) Filed: May 18, 2018

Related U.S. Application Data
(63) Continuation of application No. 14/732,147, filed on Jun. 5, 2015, now abandoned.
Application No. 14/732,147 is a continuation of application No. 14/089,118, filed on Nov. 25, 2013, now abandoned.
Application No. 14/089,118 is a continuation of application No. 11/718,362, filed on Nov. 4, 2008, now abandoned.
(60) Provisional application No. 60/646,592, filed on Jan. 26, 2005.
(60) Provisional application No. 60/623,771, filed on Nov. 1, 2004.

(51) Int. Cl. C12Q 1/689; G16H 50/20; C12Q 1/6883; G06F 19/24

(52) U.S. Cl. CPC C12Q 1/689; Y02A 90/24; Y02A 90/22; Y02A 90/26; G16H 50/20; G06F 19/24; C12Q 1/6883; C12Q 2600/16; C12Q 2600/158; C12Q 2600/112

(57) ABSTRACT

The present invention relates to methods and compositions for diagnosing, monitoring, prognosticating, analyzing, etc., polymicrobial diseases. The present invention also relates to the microbial community present in the digestive tract and lumen in normal subjects, and subjects with digestive tract diseases, especially diseases of the colon, such as inflammatory bowel disease, including ulcerative colitis, Crohn's syndrome, and pouchitis. The present invention especially relates to compositions and methods for diagnosing and prognosticating the mentioned diseases and conditions, e.g., to determine the presence of the disease in a subject, to determine a therapeutic regimen, to determine the onset of active disease, to determine the predisposition to the disease, etc.

### COMPOSITIONS AND METHODS FOR DIAGNOSING COLON DISORDERS

This Application is a Continuation of U.S. application Ser. No. 14/089,118, filed Nov. 25, 2013, which is a Continuation of U.S. application Ser. No. 11/718,362, filed Nov. 4, 2008, which is a 35 U.S.C. § 371 National Stage Application of International Application No. PCT/US2005/039887, filed Nov. 1, 2005, which claims the benefit of priority to U.S. Provisional Application No. 60/623,771, filed Nov. 1, 2004 and U.S. Provisional Application No. 60/646,592, filed Jan. 26, 2005, each of which are hereby incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MTBI_002_04US_SeqList.txt, date recorded: Jun. 5, 2015, file size ≈95 kilobytes).

### BACKGROUND OF THE INVENTION

Ulcerative Colitis and Crohn's disease are chronic inflammatory diseases of the colon and rectum. Although corticosteroids, aminosalicylates, and immunomodulators have provided some benefit in treatment of ulcerative colitis, restorative proctocolectomy ileal-pouch anal anastamosis (RP/IPAA) remains the gold standard for management of chronically active and steroid-refractory disease. The most common and debilitating complication of IPAA is symptomatic inflammation of the ileal reservoir, or pouchitis. Prior studies have demonstrated a significant decrease in quality of life (IBDQ and SF-36) when RP/IPAA is complicated by pouchitis. The incidence of pouchitis is between 30-50% up to 5 years postoperatively, with the majority of initial cases in the first 3-6 months. Clinically, pouchitis is characterized by increased stool frequency, fecal urgency, rectal bleeding, and malaise. However, the diagnosis of pouchitis is a combination of specific clinical, endoscopic, and histologic criteria. There is much debate as to whether pouchitis is an extension of the Ulcerative Colitis or a distinct disease entity. There has been no data to strongly favor either. Although many theories have been proposed, the precise mechanism of disease in pouchitis remains elusive. The dramatic clinical response to antibiotics in pouchitis suggests that microflora may play a causal role. Despite an 80% initial response to antibiotics, 60% of patients have recurring episodes of pouchitis and up to 30% of patients develop chronic symptomatic pouchitis. There have been no studies to date identifying any specific micro floral pattern or organism in the pathogenesis of pouchitis. In this study, we introduce Amplicon Length Heterogeneity, a novel culture-independent technique for detailed microfloral characterization in pouchitis.

### DESCRIPTION OF THE DRAWINGS

FIG. 1. Histogram of Crohns tissue and lumen ALH Fingerprints.

FIG. 2. Histogram of Ulcerative Colitis tissue and lumen ALH Fingerprints.

FIG. 3. Principal coordinate analysis (PCO) of Crohns and Ulcerative Colitis ALH Fingerprints.

FIG. 4. Histogram of normal Pouch and Pouchitis tissue ALH Fingerprints.

FIG. 5. Principle coordinate analysis (PCO) of Pouchitis ALH Fingerprints,

FIG. 6. Identification of peaks in normal Pouch and Pouchitis Histogram.

### DESCRIPTION OF THE INVENTION

The present invention relates to methods and compositions for diagnosing, monitoring, prognosticating, analyzing, etc., polymicrobial diseases. A polymicrobial disease is a disease or condition that is associated with the presence of at least two different microbes, including, e.g., associations between bacteria-bacteria, virus-virus, parasite-parasite, bacteria-virus, bacteria-parasite, and virus-parasite. A preferred method of determining the microbial community present in a polymicrobial disease is amplicon length heterogeneity (“ALH”).

Examples of polymicrobial diseases include, but are not limited to, e.g., co-infection of Borrelia and Ehrlichia in Lyme borreliosis; mixed viral-bacterial infections during influenza pandemics; respiratory diseases; gastroenteritis; conjunctivitis; keratitis; hepatitis; periodontal diseases; genital infections; intra-abdominal infections; inflammatory bowel diseases; urinary tract infections; necrotizing soft-tissue infection.

The present invention also relates to the microbial community present in the digestive tract and lumen in normal subjects, and subjects with digestive tract diseases, especially diseases of the colon, such as inflammatory bowel disease, including ulcerative colitis, Crohn's syndrome, and pouchitis, The present invention especially relates to compositions and methods for diagnosing, prognosticating, and/or monitoring the disease progression of the mentioned diseases and conditions, e.g., to determine the presence of the disease in a subject, to determine a therapeutic regimen, to determine the onset of active disease, to determine the predisposition to the disease, to determine the course of the disease, etc.

The present invention provides methods for diagnosing and monitoring the disease progression of inflammatory bowel diseases, such as ulcerative colitis, Crohn's disease, or pouchitis, comprising determining the presence or absence of microbes, such as bacteria, in a colon or lumen sample obtained from said subject. The invention is not limited to how the determination is carried out; any suitable method can be used. The term “microbe” includes viruses, bacteria, fungi, and protists. Although the disclosure below may be written in terms of bacteria, any microbe can be used.

The present invention relates to any composition or method which is suitable for detecting a microbial community in a sample (e.g., from a subject having a polymicrobial disease), such as a digestive tract, lumen, or stool sample. A lumen sample is from interior of the intestine,

Any marker which is suitable for identifying and distinguishing a microbial type can be utilized in accordance with present invention. These methods can involve detection of nucleic acid (e.g., DNA, RNA, mRNA, tRNA, rRNA, etc), protein (e.g., using antibodies, protein binding reagents), and any other bio-molecule (e.g., lipid, carbohydrates, etc) that is useful for specifically determining the presence or absence of bacteria in a sample. Any variable indicator or non-coding segment (e.g., repetitive elements, etc.) can also be used, as well as indicator genes. ITS regions can b e utilized in fungi.

Standard culture methods can also be utilized, where bacteria and other microorganisms are identified by culturing them on a media, e.g., using a selective media (e.g., comprising a bacteria-specific carbon source) and/or where microorganisms are identified by their growth characteristics, morphology, and other criteria typically used to determine cell identity and phylogenetic classification. Any of these methods can also be used in combination with cytological and histological methods, where biopsy samples or cultured samples can be stained and visualized (e.g., by sectioning, or by mounting on a slide or other carrier).

As mentioned, the compositions and methods are useful for diagnostic and prognostic purposes associated with polymicrobial diseases, such as inflammatory bowel diseases, including ulcerative colitis, Crohn's disease, and pouchitis. The markers and fingerprints can be utilized to diagnose the diseases, and distinguish them from other diseases of the digestive tract. They can also be used for assessing disease status, severity, and prognosis, alone, or in combination with other tests. For example, the markers can be used in conjunction with the Crohn's disease activity index (CDAI) or the criteria of Trulove and Witts for assessing disease activity in ulcerative colitis. The information about microorganismal status can also be used to determine when to initiate drug treatment or other therapeutic regimens.

The methods and compositions can also be used to monitor the course of the disease in a subject under treatment or monitor the progression of the disease, irrespective of the treatment regimen. For example, patients with inflammatory bowel syndromes may show spontaneous or drug-induced remissions. To monitor the course of the remission and determine when the disease is active, samples can be obtained periodically, and assayed to determine the appearance of the particular microbial markers or fingerprints in the intestinal tissue, lumen, colonic wash, mucosal samples, or stool.

Assessment of the microbial community can be performed on any sample obtained from a subject, including from lumen, colonic wash, intestinal tissue, intestinal mucosa, gastric tissue, gastric mucosa, stool, etc. Samples can be obtained from any part of the digestive tract, especially the small and large intestines. The large intestine or colon is the part of the intestine from the cecum to the rectum. It is divided into eight sections: the cecum, the appendix, the ascending colon, the transverse colon, the descending colon, the sigmoid colon, the rectum, and the anus. A colonic wash is the fluid left in the intestine after a subject has been given a laxative. The intestinal mucosa is the surface lining of the intestinal tract. Subjects include, e.g., animals, humans, nonhuman primates, mammals, monkeys, livestock, sheep, goats, pigs, pets (e.g., dogs, cats), small animals, reptiles, birds, etc.

Any suitable method can be utilized to obtain samples from the intestine. Endoscopic biopsy is common method in which a fiber optic endoscope is inserted into the gastrointestinal tract through a natural body orifice. The lining of the intestine is directly visualized and a sample is pinched off with forceps attached to a long cable that runs inside the endoscope. Suitable endoscopes and instruments for removing biopsy samples are well known, and include those disclosed in, e.g., U.S. Pat. Nos. 6,632,182, and 6,443,909.

Table 3 summarizes bacteria which have been detected in mucosa tissue and lumen from control subjects, and subjects having Crohn's disease or ulcerative. Table 5 summarizes bacteria which have been detected in the mucosa and lumen of subjects having pouchitis and pouchitis control (subjects with restorative procto colectomy, but without post-operative complications). PCR amplicons were cloned and sequenced from these samples. Briefly, DNA was extracted from each pooled sample. The pooled DNA from mucosa comprised DNA from mucosal and other gastrointestinal cells, as well as the bacteria. The first two variable regions of the 16S ribosomal RNA were amplified using universal Eubacterial primers. Subsequently, the amplification mixture was separated and characterized on a fingerprinting gel. The resulting picture of the gel or tabular compilation of the data (see, e.g., Tables 1, 2, and 4)—comprising discrete, individual bands (PCR amplicons)—can be referred to as the “ALH fingerprint.” The ALH fingerprint can be further characterized by identifying the length of the individual replicons that comprise it and/or their specific nucleotides sequences. Amplicons from the microbial community can then be cloned and sequenced, where the sequence is correlated with a particular bacterial group, species, or strain. Using this method, the abundance of the clones from each species is proportional to their abundance in the corresponding community, and can be correlated to peaks in the ALH fingerprint. The sequence data can be used to search the Ribosomal database (RDP) using a standard sequence search tool (Megablast) available from the National Center for Biotechnology Information (NCBI) at NIH. See, e.g., Cole J R, Chai B, Marsh T L, Farris R J, Wang Q, Kulam S A, Chandra S, McGarrell D M, Schmidt T M, Garrity G M, Tiedje J M. The Ribosomal Database Project (RDP-II): previewing a new autoaligner that allows regular updates and the new prokaryotic taxonomy. Nucleic Acids Res 2003 Tan 1; 31(0:442-3. The RDP number obtained from the search results can be parsed using a custom PERL script to classify the Division, Subdivision, Group, and Subgroup of each clone, and the results can be tabulated, and imported into EXCEL or other suitable databases.

### Nucleic Acid Detection Methods

Detection methods have a variety of applications, including for diagnostic, prognostic, forensic, and research applications. To accomplish nucleic acid detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a polynucleotide (e.g., copies of a ribosomal RNA) present in a sample. As explained in more detail below, any suitable method can be used, including, but limited to, ALH, PCR, nucleotide sequencing, Southern blot, and or DNA microarrays (e.g., where a microarray comprises a plurality of sequences specific for one or more bacteria of the present invention).

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene cloning and Analysis; Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; W097/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/1031 5), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of polynucleotide or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

The present invention provides methods for diagnosing or prognosticating ulcerative colitis, Crohn's disease, or pouchitis, or in a subject, comprising, one or more of the following steps in any effective order, e.g., contacting a gastrointestinal tissue or lumen sample comprising nucleic acid with a polynucleotide probe which is specific for at least one bacteria under conditions effective for said probe to hybridize specifically with said nucleic acid, and detecting hybridization between said probe and said nucleic acid, wherein the presence of one or more bacteria selected from the following group said bacteria indicates the disease presence or the disease status of ulcerative colitis, Crohn's disease, or Pouchitis. The method can further comprise obtaining a colon sample, e.g., by endoscopic biopsy, and/or extracting the nucleic acid from the sample.

The phrases “specific for” or “specific to” a microbe has a functional meaning that indicates the probe (or antibody if it used in a protein context) can be used to identify the presence of the target microbe in a sample and distinguish it from non-target microbe. It is also specific in the sense that it can be used to detect target microbe above background noise (“non-specific binding”). This same definition is also applicable to a polynucleotide or antibody probe. Probes can also be described as being specific for a sequence, where a specific sequence is a defined order of nucleotides (or amino acid sequences, if it is a polypeptide sequence) that occurs in the polynucleotide.

The phrase “hybridize specifically” indicates that the hybridization between single-stranded polynucleotides is based on nucleotide sequence complementarity. The effective conditions are selected such that the probe hybridizes to a pre-selected and/or definite target nucleic acid in the sample. For instance, if detection of a polynucleotide for a ribosomal RNA is desired, a probe can be selected which can hybridize to the target ribosomal RNA under high stringent conditions, without significant hybridization to other non-target sequences in the sample. For example, the conditions can be selected routinely which require 100% or complete complementarity between the target and probe.

Contacting the sample with probe can be carried out by any effective means in any effective environment. It can be accomplished in a solid, liquid, frozen, gaseous, amorphous, solidified, coagulated, colloid, etc, mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved, such as hybridization between a probe and its target, or antibody binding to a target protein. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

For detecting the presence of a probe specifically hybridized to a target, any suitable method can be used. For example, polynucleotides can be labeled using radioactive tracers such as ³²P, ³⁵S, ³H, or ¹⁴C, to mention some commonly used tracers. Non-radioactive labeling can also be used, e.g., biotin, avidin, digoxigenin, antigens, enzymes, or substances having detectable physical properties, such as fluorescence or the emission or absorption of light at a desired wavelength, etc.

Any test sample in which it is desired to identify the presence or absence of bacteria can be used, including, e.g., blood, urine, saliva, lumen (for extracting nucleic acid, see, e.g., U.S. Pat. No. 6,177,251), stool, swabs comprising tissue, biopsied tissue, tissue sections, cultured cells, intestinal wash, colonic wash, intestinal mucosa, etc

The results for any of the assays mentioned herein (including the assays in other sections below) can be with respect to a control sample. For example, an increase or decrease can be with respect (in comparison) to a normal lumen or mucosa sample. The normal sample can be from the same patient, but from an unafflicted region or period (e.g., when the patient is in remission). It can also be from a standard value that is calculated based on a normalized population of individuals. Standard statistics can be utilized to determine whether the values are significant.

The present invention also provides methods for nucleic acid fingerprinting the community of microbes present in a sample, e.g., using universal primers to the microorganisms in question, whether they be Eubacteria, Archaebacteria, Fungi, or Protists. Since each sample contains a distinctive population of microbes that is representative of the disease, sampling the nucleic acids from the microbes can produce a distinctive array of polynucleotide fragments associated with the disease. These can be presented by any physical characteristic, including size, sequence, mobility, molecular weight (e.g., using mass spectroscopy), etc. Any fingerprinting method can be used, including, e.g., AFLP, ALH, LH-PCR, ARISA, RAPD, etc. Tables 1, 2, and 4 show the frequency of amplicons in various control and disease samples. Although one particular amplicons may not be diagnostic of the condition 100% of the time, using multiple amplicons increases the diagnostic certainty. Moreover, when a condition is being monitored, it may be advantageous to monitor a complex fingerprint (such as shown in Table 1) a it differs from one sampling time to another.

Along these lines, the present invention provides method for diagnosing, prognosticating, or monitoring the disease progression of a polymicrobial disease (e.g., an inflammatory bowel disease, such as ulcerative colitis, pouchitis, or Crohn's disease), comprising one or more of the following steps in any effective order, e,g., performing an amplification reaction on a sample comprising nucleic acid with at least two polynucleotide probe primers which are effective for amplifying the microbial community present in said sample, and detecting the reaction products of said amplification reaction, whereby said reaction products comprise a pattern that indicate the presence of the disease or the disease status.

By “disease status,” it is meant the relative condition of the disease as compared to its condition at a previous time. For example, when sample reaction products differ (e.g., in quantity or size) from a period of disease severity, this would indicate that the disease status of the subject had changed. The reaction products may show a difference before the subject actually manifests symptoms of the disease, and therefore can be used prognostically to predict a relapse, Similarly, a change in the reaction products can also indicate that the disease is improving and/or responding to a treatment regime.

The term “amplification” indicates that the nucleic acid sequences are increased in copy number to an amount or quantity at which they can be detected. Amplification can be carried out conventionally, using any suitable technique, including polymerase chain reaction (PCR), NASBA (e.g., using T7 RNA polymerase), LCR (ligation chain reaction), LH-PCR, ARISA.

Total nucleic can be extracted from a sample, or the sample can be treated in such a way to preferentially extract nucleic acid only from the microbes that are present in it. DNA extractions can be performed with commercially available kits, such as the Bio101 kit from Qbiogene, Inc, Montreal, Quebec, To prevent contamination by multiple samples during the homogenization process of a sample, each individual sample can be processed separately and completely leading to high yield DNA extractions.

In certain embodiments of the present invention, ribosomal RNA (“rRNA”) can be used to distinguish and detect bacteria. For example, bacterial ribosomes are comprised of a small and large subunit, each which is further comprised of ribosomal RNAs and proteins. The rRNA from the small subunit can be referred to as SSU rRNA, and from the larger subunit as LSU rRNA. A large number of rRNAs have been sequenced, and these are publicly available in various accessible databases. See, e.g., Wuyts et al., The European database on small subunit ribosomal RNA, Nucleic Acids Res., 30, 183-185, 2002; Cole et al., The Ribosomal Database Project (RDP-II): previewing a new autoaligner that allows regular updates and the new prokaryotic taxonomy. Nucleic Acids Res., 31(1): 442-3, 2003. See, also, http://rdp.cme.msu.edu/html/ accessed on Jun. 14, 2004. Any rRNA can be used as a marker, including, but not limited to, 16S, 23S, and 5S.

Primer sequences to rRNA can be designed routinely to detect specific species of bacteria, or to detect groups of bacteria, e.g., where a conserved sequence is characteristic of a bacterial group. ALH-PCR can be accomplished routinely, e.g., using a fluorescently labeled forward primer 27F (5′-[6FAM] AGAGTTTGATCCTGGCTCAG3′) (SEQ ID NO:37) and unlabeled reverse primer 338R′ (5′- GCTGCCTCCCGTAGGAGT-3′) (SEQ ID NO:38). Both primers are highly specific for Eubacteria (Lane, D. J., 168/23S rRNA Sequencing, in Nucleic Acid Techniques in Bacterial Systematics, E.S.a.M. Goodfellow, Editor. 1991, John Wiley & Sons Ltd: West Sussex, England. p. 115-175).

Primers can also be utilized which amplify the corresponding region in Archae (Burggraf, S., T. Mayer, R. Amann, S. Schadhauser, C. R. Woese and K. O. Stetter, Identifying Members of the Domain Archaea with rRNA-Targeted Oligonicleotide Probes. App. Environ. Microbiol., 1994. 60: p. 3112-3119), Eukaryotes (Rowan, R. and D. A. Powers, Ribosomal RNA sequences and the diversity of symbiotic dinoflagellates (zooxanthellae). Proceedings of the National Academy of Sciences, USA, 1992. 89: p. 3639-3643), and Fungi (Borneman, J. and J. Hartin, PCR primers that amplify fungal rRNA genes from Environmental Samples. App. Environ. Microbiol., 2000. 66(10): p. 4356-4360).

Primers can be selected from any nucleic acid of the infectious agent, including from rRNA, tRNA, genomic DNA, etc. The primers can be to variable regions, helices, conserved regions, etc.

Selected primers can be utilized in amplicon length heterogeneity (“ALH”) to generate fingerprints that characterize the bacterial community (Ritchie, N. J., et al., Use of Length Heterogeneity PCR and Fatty Acid Methyl Ester Profiles to Characterize Microbial Communities in Soil, Applied and Environmental Microbiology, 2000, 66(4): p. 1668-1675; Suzuki, M., M. S. Rappe, and S. J. Giovannoni, Kinetic bias in estimates of coastal picoplankton community structure obtained by measurements of small-subunit rRNA gene PCR. amplicon length heterogeneity. Applied and Environmental Microbiology [Appl. Environ Microbiol.], 199.64(11): p. 45224529; Litchfield, C. D. and P. M. Gillevet, Microbial diversity and complexity in hypersaline environments: A preliminary assessment. Journal of Industrial Microbiology & Biotechnology [J. hid. Microbiol. Biotechnol.]. 2002. 28(1): p. 48-55; Mills, D. K., et al., A Comparison of DNA Profiling Techniques for Monitoring Nutrient Impact on Microbial Community Composition during Bioremediation of Petroleum Contaminated Soils. J. Microbiol. Method, 2003. 54: p. 57-74).

Individual primers can be utilized or a mixture, e.g., comprising degenerate sequences, sequences from one or more group, multiplex reaction where different groups are assessed using primers labeled with different fluorescent tags etc.

The reaction products (i.e., the fragments which are detected after the amplification reaction) can be analyzed by statistical analysis, such as PCQ analysis (see Examples) to determine which products are diagnostic of the disease.

### Polypeptide Detection

The present invention also provides compositions and methods for detecting polypeptides and other biomolecules that are characteristic of the microbial population. For example, the present invention provides methods for diagnosing or prognosticating ulcerative colitis, pouchitis, or Crohn's disease comprising:, one or more of the following steps in any effective order, e.g., contacting a sample comprising protein with an antibody which is specific for a bacteria under conditions effective for said antibody to specifically bind to said bacteria, and detecting binding between said antibody and said bacteria.

Polypeptides can be detected, visualized, determined, quantitated, etc. according to any effective method. Useful methods include, e.g, but are not limited to, immunoassays, RIA. (radioimmunassay), ELISA, (enzyme-linked-immunosorbent assay), immunoflourescence, flow cytometry, histology, electron microscopy, light microscopy, in situ assays, immunoprecipitation, Western blot, etc.

Immunoassays may be carried in liquid or on biological support. For instance, a sample (e.g., blood, lumen, urine, cells, tissue,cerebral spinal fluid, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled bacteria specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads

One of the many ways in which a bacteria specific antibody can be detectably labeled is by linking it to an enzyme and using it in an enzyme immunoassay (ETA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, .beta.- galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The present invention also relates to preventing and/or treating inflammatory bowel conditions in a subject in need of, comprising administering lantibiotics, as well as other antibacterial compounds, which are produced by bacteria in the digestive tract of normal individuals. A probiotic approach can be used, where bacteria that produce these compounds are administered, instead of providing the compounds in purified forms.

As described in detail above, the microbial community of subjects with inflammatory bowel conditions is perturbed. These perturbations can have profound consequences on the health of the subject. Certain bacteria, such as Ruminococcus sp. produce lantibiotics that have protective and antibacterial effects on pathogenic bacteria. For example, it is shown above in Table 5 above that R. gnavus is reduced in subjects having Crohn's disease and ulcerative colitis. R. gnavus produces a lantibiotic (RumA) that is active against pathogenic bacteria. The reduction in the R. gnavus community in these subjects can result in the growth of deleterious bacteria (such as pathogenic bacteria) that in turn is associated with an inflammatory response. Conversely, certain bacteria associated with these inflammatory bowel conditions can produce lantibiotics that inhibit beneficial bacteria such as Lactobacillus species. By providing the lantibiotic (either in purified or as a probiotic), subjects with these conditions can be treated. Any lantibiotic produced by a bacteria described herein can be utilized to prevent and/or treat inflammatory bowel conditions. The RDP group, the representative genus, or the species of the bacteria listed in Tables 3 and 5 can be utilized for diagnostic, prognostic, and disease monitoring purposes in accordance with the present invention. For instance, an increase in a Moraxella osloensis was observed in Crohns mucosa in comparison to control mucosa. This information was obtained from a sequenced clone originating in the mucosa of a Crohns patient. Sequence searching of the RDP database Version 8.1 (Cole J R, Chai B, Marsh T L, Farris R J, Wang Q, Kulam S A, Chandra S, McGarell D M, Schmidt T M, Garrity G M, Tiedje J M. The Ribosomal Database Project (RDP-II): previewing a new autoaligner that allows regular updates and the new prokaryotic taxonomy. Nucleic Acids Res 2003 Jan. 1; 31(1):442-3) (see, e.g., world wide web at rdp8.cme.msu.edu/html) indicated that it was a member of the Pseudomonas_and_relatives RDP group, and more precise sequence analysis assigned it to the Moraxella genus. For the purposes of the present invention, the RDP group alone can be used as the indicator of disease status. Thus, in the above-mentioned example, classifying a bacteria as a member of the Pseudomonas_and_relatives RDP group (irrespective of its genus or species classification) is sufficient to indicate that the patient harboring the bacteria in their intestinal mucosa is more likely to be afflicted with Crohns disease, or to be regressing from a temporary remission. One or more groups can be used diagnostically. Therefore, with respect to the example above, determining that a patient's microbial community comprises both Pseudomonas_and_relatives and Acidovorax_Group bacteria indicates the existence of Crohns disease. Similar analysis can be made for all the RDP groups disclosed in Tables 3 and 5. Although not all permutations may be disclosed in the application, they can be routinely chosen from Tables 3, 5, and the appended claims.

For any given clone isolated from a subject suspected of having Crohns, Ulcerative colitis, or Pouchitis, a SSU rRNA sequence 97% identity to a known species is generally sufficient for it to be classified as that species. Similarly, about 95% identity is generally sufficient for genus and RDP group classification. Identity was determined using the BLAST algorithm (Tatusova, T. A., & Madden, T. L. (1999). BLAST 2 Sequences, a New Tool for Comparing Protein and Nucleotide Sequences. FEMS Microbiology Letters, 174, 247-250; Altschul, S. F., Madden, T. L., Schaffer, A. A., Zhang, J., Zhang, Z., Miller, W., et al. (1997). Gapped BLAST and PSI-BLAST; a new generation of protein database search programs. Nucleic acids research, 1997 Sep. 1, 25(0:3389-402; Wheeler, D. L., Chappey, C., Lash, A. E., Leipe, D. D., Madden, T. L., Schuler, G. D., et al. (2000). Database resources of the National Center for Biotechnology Information. Nucleic Acids Res, 28(1), 10-14.).

The present invention provides methods diagnosing, prognosticating, and/or monitoring disease progression of Crohn's disease ulcerative colitis, or pouchitis, or in a subject, comprising: contacting a colonic mucosal tissue sample comprising nucleic acid with a polynucleotide probe which is specific for at least one bacteria under conditions effective for said probe to hybridize specifically with said nucleic acid, and detecting hybridization between said probe and said nucleic acid, wherein an increase, as compared to a normal mucosa sample, of one or more bacteria species or RDP group selected from the following indicates the disease presence or the disease status: a) Crohn's disease:

Morexella sp. of Pseudomonas group; Comamonas sp. of the Acidovorax_Group; or Cryseobacterium sp. of the Cytophaga_Group_I (where the RDP group and/or genus and/or genus_species can be used); b) ulcerative colitis: Morexella sp. of Pseudomonas_and_Relatives; Comamonas sp. of the Acidovorax_Group; Clostridium sp. of the Clostridium botulinum_Group; or Enterococus sp. of the Enterococcus_Group (where the RDP group and/or genus and/or genus_species can be used); or c) pouchitis (compared to normal pouch): Ruminococus sp. of Clostridium_Coccoides_Group; Escherichia coli and Shigella sp. of the Enterics and Relatives group; or Fusobacterium sp. of the Fusobacteria_{—} Group (where the RDP group and/or genus and/or genus_species can be used).

The present invention also provides methods for diagnosing, prognosticating, and/or monitoring disease progression of Crohn's disease, ulcerative colitis, or pouchitis, comprising: contacting a colonic mucosal tissue sample comprising nucleic acid with a polynucleotide probe which is specific for at least one bacteria under conditions effective for said probe to hybridize specifically with said nucleic acid, and detecting hybridization between said probe and said nucleic acid, wherein a decrease, as compared to a normal mucosa sample, of one or more bacteria selected from the following group said bacteria indicates the disease presence or the disease status: a) Crohn's disease: Bacteroides sp. of the Bacteroides Group; Propionibacterium sp. of the Propionibacterium Group; or Ruminoccocus sp. of the Clostridium Coccoides Group (where the RDP group and/or genus and/or genus_species can be used); b) ulcerative colitis: Bacteroides sp. of the Bacteroides Group; Propionibacterium sp. of the Propionibacterium Group; or Ruminoccocus sp. of the Clostridium Coccoides Group (where the RDP group and/or genus and/or genus_species can be used); c) pouchitis: Bacteroides sp. of the Bacteroides Group; Propionibacterium sp. of the Propionibacterium Group (where the RDP group and/or genus and/or genus_species can be used).

The present invention also provides methods for diagnosing, prognosticating, and/or monitoring disease progression of Crohn's disease ulcerative colitis, or pouchitis, or in a subject, comprising: determining the presence of one or more of the following bacteria in a colonic mucosal tissue from a subject having Crohn's disease, ulcerative colitis, or pouchitis: a) Crohn's disease: Morexella sp. of Pseudomonas group;

Comamonas sp. of the Acidovorax_Group; or Cryseobacterium sp. of the Cytophaga_Group_I (where the RDP group and/or genus and/or genus_species can be used); b) ulcerative colitis: Moroxella sp. of Pseudomonas_and_Relatives; Comamonas sp. of the Acidovorax_Group; Clostridium sp. of the Clostridium botulinum_Group; or Enterococus sp. of the Enterococcus_Group (where the RDP group and/or genus and/or genus_species can be used); c) pouchitis (compared to normal pouch): Ruminococus sp. of Clostridium_Coccoides_Group; Escherichia coli and Shigella sp. of the Enterics and Relatives group; or Fusobacterium sp. of the Fusobacteria_{—} Group (where the RDP group and/or genus and/or genus_species can be used).

The present invention also provides methods for diagnosing, prognosticating, and/or monitoring disease progression of Crohn's disease, ulcerative colitis, or pouchitis, comprising: determining the absence of one or more of the following bacteria in a colonic mucosal tissue from a subject having Crohn's disease, ulcerative colitis, or pouchitis: Crohn's disease: Bacteroides sp. of the Bacteroides Group; Propionibacterium sp. of the Propionibacterium Group; or Ruminoccocus sp. of the Clostridium Coccoides Group (where the RDP group and/or genus and/or genus_species can be used); b) ulcerative colitis: Bacteroides sp. of the Bacteroides Group; Propionibacterium sp. of the Propionibacterium Group; or Ruminoccocus sp. of the Clostridium Coccoides Group (where the RDP group and/or genus and/or genus_species can be used); c) pouchitis: Bacteroides sp. of the Bacteroides Group; or Propionibacterium sp. of the Propionibacterium Group (where the RDP group and/or genus and/or genus_species can be used).

The present invention also provides method for diagnosing, prognosticating, and/or monitoring disease progression of Crohn's disease or ulcerative colitis, in a subject, comprising: contacting a lumen sample comprising nucleic acid with a polynucleotide probe which is specific for at least one bacteria under conditions effective for said probe to hybridize specifically with said nucleic acid, and detecting hybridization between said probe and said nucleic acid, wherein an increase, as compared to a normal lumen. sample, of one or more bacteria selected from the following indicates the disease presence or the disease status: a) Crohn's disease: Bacteriodes sp. of the Bacteriodes_Group; or Chryseobacterium sp. of the Cytophaga_Group_I (where the RDP group and/or genus and/or genus_species can be used); or b) ulcerative colitis: Bacteriodes sp. of the Bacteriodes_Group; or Chryseobacterium sp. of the Cytophaga_Group_I (where the RDP group and/or genus and/or genus_species can be used).

The present invention also provides methods for diagnosing, prognosticating, and/or monitoring disease progression of Crohn's disease or ulcerative colitis in a subject, comprising: contacting a lumen sample comprising nucleic acid with a polynucleotide probe which is specific for at least one bacteria under conditions effective for said probe to hybridize specifically with said nucleic acid, and detecting hybridization between said probe and said nucleic acid, wherein a decrease, as compared to a normal lumen sample, of Acinetobacter sp. or Moraxella sp. of the Pseudomonas and relatives group indicates that said subject has Crohn's disease or ulcerative colitis (where the RDP group and/or genus and/or genus_species can be used).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Sample Collection and DNA Extraction:

Endoscopic mucosal tissue samples were collected from the terminal ileum, cecurn+ascending colon, transverse colon, sigmoid colon and the rectum of patients with IBD and Pouchitis as well as healthy controls undergoing the colonoscopy. Some of the tissue samples were washed in saline prior to analysis to remove non-adherent bacteria (washed vs. unwashed samples). Retained lumen samples were also collected via the endoscope at the time of procedure. The samples were fingerprinted for bacterial patterns in 4 control, 2 UC, 4 CD and 3 patients with pouchitis, and 5 patients with pouch without pouchitis using the ALH methodology. The DNA extractions were performed using the Bio101 soil kit from Qbiogene, Inc, Montreal, Quebec according to the manufacturers instructions. These ALH amplicons were pooled, then cloned and sequenced to identify the bacterial components that were indicative of the disease state.

### Amplicon Length Heterogeneity (ALH) Fingerprinting:

Amplicon Length Heterogeneity (ALH) Fingerprinting: ALH is a technique of bacterial fingerprinting see Ritchie, N J., et al., Use of Length Heterogeneity PCR and Fatty Acid Methyl Ester Profiles to Characterize Microbial Communities in Soil. Applied and Environmental Microbiology, 2000. 66(4): p. 1668-1675; Suzuki, M., M. S. Rappe, and S J. Giovannoni, Kinetic bias in estimates of coastal picoplankton community structure obtained by measurements of small-subunit rRNA gene PCR amplicon length heterogeneity. Applied and Environmental Microbiology [Appl. Environ. Microbiol.]. ALH is a PCR-based analysis which can distinguish different organisms based on natural variations in the length of 16S ribosomal DNA sequences. Purified DNA (10 ng) was amplified with PCR by using a fluorescently-labeled forward primer 27F (5’-[6FAM] AGAGTTTGATCCTGGCTCAG-3′ (SEQ ID NO: 37)) and unlabeled reverse primer 338R′ (5′-GCTGCCTCCCGTAGGAGT-3′ (SEQ ID NO: 38)). Both primers are highly specific for eubacteria, Alternatively, we have primers that amplify the corresponding region in Archae see Burggraf, S., T, Mayer, R, Amann, S, Schadhauser, C. R, Woese and K. O. Stetter, Identifying Members of the Domain Archaea with rRNA-Targeted Oligonucleotide Probes. App. Environ. Microbiol., 1994. 60: p. 3112-3119. We have recently optimized primers specific to the ITS of fungi and demonstrated that these region of the rRNA operon is more informative that the SSU rRNA see Borneman, J. and J. Hartin, PCR primers that amplify fungal rRNA genes from Environmental Samples. App, Environ. Microbiol, 2000. 66(10): p. 4356-4360. The reactions were performed using 50-ul (final volume) mixtures containing 1× PCR buffer, 0.6% bovine serum albumin, 1.5 mM MgCl2, each deoxynucleoside triphosphate at a concentration of 0.2 mM, each primer at a concentration of 0.2 uM, and 2 U of Taq DNA polymerase. Initial denaturation at 94 C for 3 min was followed by 25 cycles consisting of denaturation at 94 C for 45 sec, annealing at 55 C for 45 s, and extension at 72 C for 2 min. There was a final extension step that consists of 72 C for 7 min. ALH samples are were stored at −20 C in the dark until used (usually less than 1 week).

The ALH PCR products were separated on the SCE9610 capillary fluorescent sequencer (Spectrumedix LLC, State Colleges, PA) and analyzed with their GenoSpectrum software package. The software converts fluorescence data into electropherograms The peaks of the electropherograms represent different populations of microflora of different sizes. All fingerprinting data was analyzed using software (Interleave 1.0, BioSpherex LLC) that combines data from several runs, interleaves the various profiles, normalizes the data, and calculates diversity indices. The normalized peak areas were calculated by dividing an individual peak area by the total peak area in that profile.

### Analysis of ALH Fingerprints:

The diversity of AUH fingerprints were analyzed using indices of Richness (R), Evenness (E) and the Shannon-Weaver diversity index (SW) in groups comparing IBD to controls, see Hughes, j.3., et al., Counting the Uncountable: Statistical Approaches to Estimating Microbial Diversity. App. Environ. Microbiol., 2001. 67(10): p. 4399-4406. IBD related parameters such as disease activity, and histologically involved and uninvolved parts of the ileum & colon were compared using the diversity indices.

These fingerprints were analyzed to determine global clustering of ALH fingerprints into presence or absence of IBD, IBD-types (CD, UC and pouchitis), disease activity, and involved and uninvolved parts of the ileum & colon (tissue state). Multidimensional Reductions Analysis [Principal Component Analysis (PCA), Principal Coordinate Analysis (PCO), Canonical Correspondence Analysis (CCA)] and Clustering Analysis was done using the Multi Variate Statistical Package (MVSP), Kovach Computing Services, Wales, UK. The following analyses will be done. Generation of dendograms by Unweighted Pair Group Method using Arithmetic Averages (UPGMA).

### Principal Component Analysis (PCA):

PCA is one of the best known and earliest ordination methods, first described by Karl Pearson (1901). Graphically, it is a rotation of a swarm of data points in multidimensional space so that the longest axis (the axis with the greatest variance) is the first PCA axis, the second longest axis perpendicular to the first is the second PCA axis, and so forth. The first few PCA axes represent the greatest amount of variation in the data set. The first two or three axes are generally expected to account for a large proportion of the variance, e.g. 50-60% or more.

### Principal Coordinates Analysis (PCO):

PCO can be viewed as a more general form of PCA. PCO can use a variety of different measures of distance or similarity. In general, the distances or similarities are measured between the cases directly, rather than the variables as in PCA. The main advantage of PCO is that many different kinds of similarity or distance measures can be used. PCO is restricted to analyzing distances or similarities that are metric and the distances used must be able to be viewed in some sensible geometrical manner e.g. a triangle.

### Canonical Corespondance Analysis (CCA):

In PCA & PCO, the data are subjected to some type of mathematical manipulation in order to reveal the most important trends. These trends are then often compared to other data relating to the same samples to determine the relationship between the two. However, in CCA, the data are directly related. CCA is a multivariate direct gradient analysis method that has become very widely used in ecology.

### Cluster Analysis:

Cluster analysis is a term used to describe a set of numerical techniques in which the main purpose is to divide the objects of study into discrete groups. These groups are based on the characteristics of the objects and it is hoped the clusters will have some sort of significance related to the research questions being asked. Cluster analysis is used in many scientific disciplines and a wide variety of techniques have been developed to suit different types of approaches. The most commonly used ones are the agglomerative hierarchical methods. Hierarchical methods arrange the clusters into a hierarchy so that the relationships between the different groups are apparent and the results are presented in a tree-like diagram called a dendrogram. The agglomerative methods used to create a dendrogram start by successively combining the most similar objects until all are in a single, hierarchical group Similarly dendograms can be created using the well established Unweighted Pair Group Method using Arithmetic Averages (UPGMA) and K-means.

Putative ALH fingerprint patterns (i.e. presence or absence of certain amplicon peaks) associated with IBD presence, disease types, disease activity, and tissue state were identified. For this purpose, we will visually inspect histograms of ALH fingerprints. To determine statistical correlations of peaks to IBD related variables, we also used multivariate analysis for large variable sets i.e. discriminate analysis and Canonical correspondence analysis. We also employed computerized data mining tools with supervised and unsupervised pattern recognition algorithms. These include C4.5, support vector machines, and self organizing maps. Hence, these analyses will be used to determine if ALH fingerprinting can distinguish between IBD related parameters (disease presence, type, activity, tissue state) and determine particular ALH patterns (presence or absence of a peak or sets of peaks) associated with IBD.

### Sequencing of ALH Clones:

The PCR product generated with primers used for ALH fingerprinting were cloned by using pGEM-T Easy Vector System II (Promega Corp., Madison, Wis.). Clones were screened assessing for inserts using alpha-complementation with X-Gal (5 bromo-4chloro-3indoyl-B-D-galactopyranoside) and IPTG (isopropyl-B-D-thiogalactopyranoside). Inserts were sequenced by using Taq dye terminator chemistry and the sequencing products were separated on a SCE9610 fluorescent capillary sequencer.

### Analysis of ALH Clone Data:

The above ALH clone sequences were compared to sequences in the RDP database to assess for patterns of microflora using a novel program (CloneID 1.0, BioSpherex LLC). The algorithm basically uses Megablast to compare the clone sequence data to the RDP database and compiles a table using the RDP numbers to correlate the identification with a hierarchical classification scheme. These same ALH clones were fingerprinted to determine the empirical ALH size and correlated with the original ALH fingerprint of sample using a second program (CloneMatch 1.0, BioSpherex, LLC).

### Results of Crohns (CD) and Ulcerative Colitis (UC) Analysis:

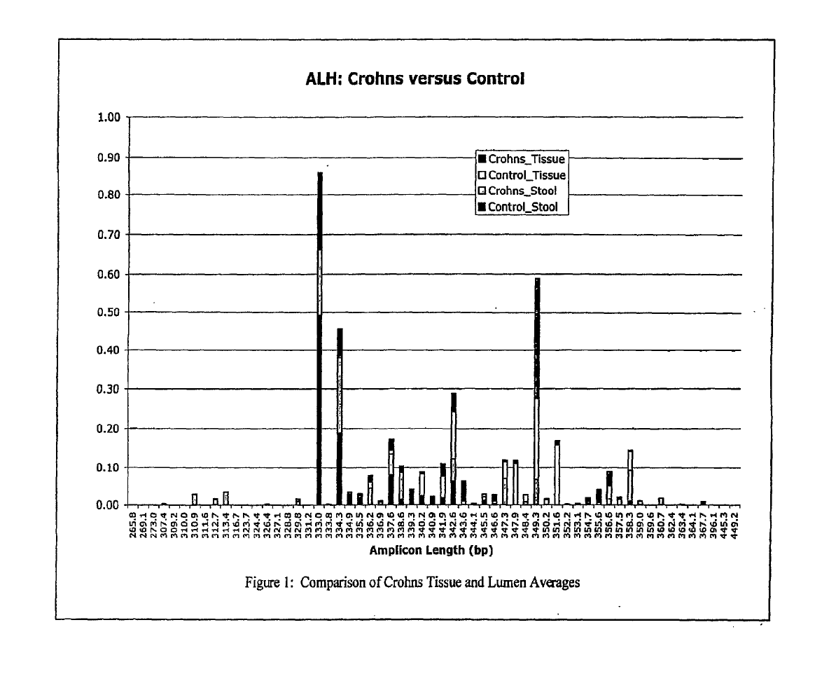

Although ALH fingerprints vary qualitatively and quantitatively between individuals, there are very distinct diagnostic patterns that can be seen from the analysis of pooled tissue (mucosa) and lumen samples. FIG. 1 is a histogram compiled from the average of the ALI-1 fingerprint from all the Crohns samples and Control samples (i.e, all individuals and all locations) showing amplicon lengths in base pairs (bp) on the x-axis and relative abundances on the y-axis. The pooled Controls Tissue samples (white bars) had very distinct ALH profile that differed dramatically from the Controls Lumen samples (black bars) indicating that there is a distinct microflora community adhering to the mucosa as a biofilm In contrast there was not a clear differentiation between the lumen and tissue microflora in Crohns disease indicating a dramatic dysbiosis in which many of the bacterial species normally in lumen are found in the biofilm. Thus, there was much more overlap between the ALH amplicons of Crohns Tissue (light grey bars) and Crohns Lumen (dark grey bars) with Control Lumen (black bars). There are diagnostic ALH amplicons that occur predominantly in the Control Lumen samples and Crohns tissue (i.e. at 333.0 bp, 334.3 bp, and 338.6), Furthermore, there are some ALH amplicons that are unique for Crohns tissue (i.e. 310.9 bp and 313.4 bp) but on average they make up a small proportion of the microflora community.

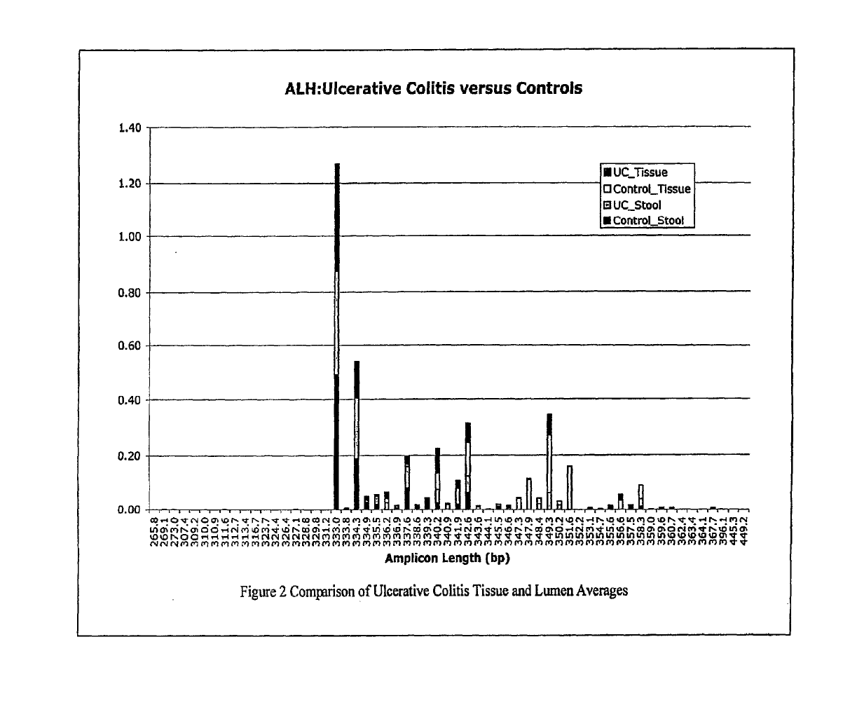

Similarly, there seems to be dysbiosis in Ulcerative colitis (UC) as the ALH profiles of UC Tissue and UC Lumen are similar to Control Lumen with the ALH profile of the Controls being very distinct (FIG. 2). Thus, there was much more overlap between the ALH amplicons of UC Tissue (light grey bars) and UC Lumen (dark grey bars) with Control Lumen (black bars). Some of the diagnostic ALH amplicons that were observed in CD (see above) are the same amplicons that are diagnostic in UC (i.e. at 333.0 bp, 334.3 bp, and 338.6 bp). Furthermore, there are distinct ALH amplicons that occur only in the UC tissue (i.e. 334.6 bp).

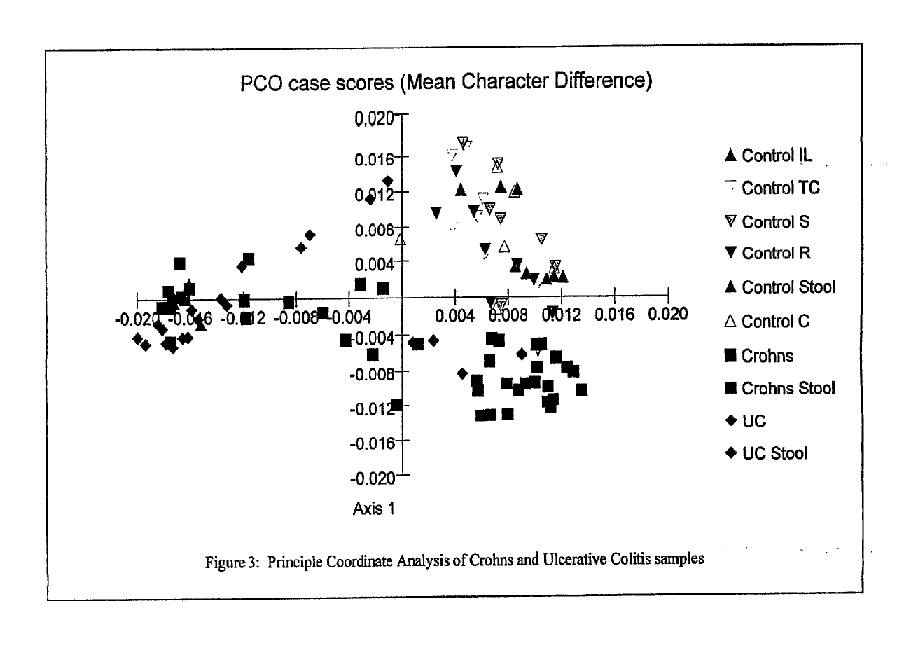

When the mean character differences for ALH profiles from Controls, CD, and UC were examined using Principle Codordinate Analysis (PCO), dramatic clustering patterns can be seen for UC and CD that is distinct from the Control samples (FIG. 3). We clearly see distinct clustering of Control ALH profiles in the 1^{st} quadrant, UC clusters in the 3^{rd} & 4^{th} quadrants boundary, and CD is mainly clustered in the 2^{nd} quadrant. It is also important to note that the lumen samples cluster in the 3^{rd} & 4^{th} quadrant associated with UC. There are also several Crohns ALH profiles that cluster in this 3rd & quadrants suggesting that there is variation in the tissue microflora of Crohns and that, in specific samples, these ALH profiles are similar to those of UC.

PCA and Canonical Correspondence Analysis demonstrates a similar clustering of healthy controls separate from CD and UC patients. The dendograms produced with UPGMA clustering using a Jacard distance measure also show the same general patterns as the PCO analysis.

We have cloned and sequenced pooled ALH amplicons from the UC, CD and healthy controls samples and these sequence data were used to identify the bacterial species associated with each disease state, Table 1 summarizes the key bacterial groups based on the RDP classification scheme that occur at a frequency of greater than 5% of the microfloral community. The data supports the ALH profiles in that the microflora found on the mucosal surface of CD and UC tissue resemble the microfloral composition of lumen in healthy individuals and that this composition differs from the microfloral composition of the controls mucosa. Specifically, members of the Pseudomonads such as Moraxella sp. and members of the Acidovorax group such as Comoamonas sp. are associated with Control lumen, CD lumen, CD mucosa, UC lumen, and UC mucosa. Additionally, members of the Cyotphaga group such as Chryseobacterium balustinum are associated with CD mucosa, CD lumen, and UC lumen. Finally, members of both of the Clostridium group (Clostridium paraputrificum) and Enterococcus (Enterococcus hirae) are also associated with UC mucosa. We also note that there is a quantitative decrease in the Bacteroides group in UC mucosa and CD mucosa compared to the Control mucosa.

In summary, we conclude that there are bacterial species that are associated in the CD biofilm and UC biofilm that are normally found in lumen and that this indicates severe dysbiosis.

### Results for Pouchitis Analysis:

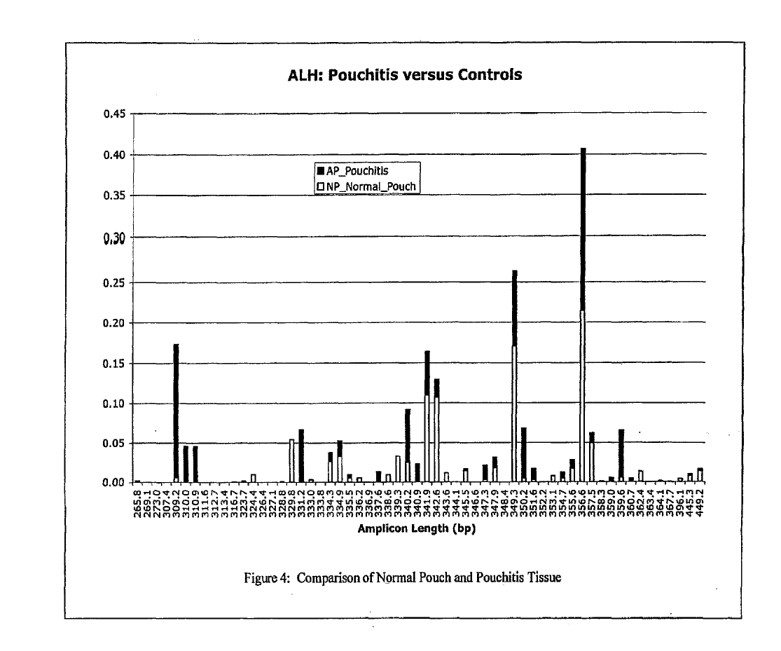

FIG. 4 is a histogram compiled from the average of the ALH fingerprint from all the Pouchitis samples (AP) and Normal pouch samples (NP), that is samples from patients with active Pouchitis (AP) and patients with a Pouch but are normal upon examination (NP). As seen in CD and UC, the pooled NP mucosa samples (white bars) had very distinct ALH profile that differed dramatically from the NP mucosa samples (black bars) indicating that there is a distinct microflora community adhering to the mucosa as a biofilm. Furthermore, the ALH amplicon profiles from the NP samples were different that healthy control patients that did not have a Pouch. There are diagnostic ALH amplicons that occur predominantly in the AP mucosa samples (i.e. at 309.2 bp, 310.0 bp, 310.9 bp, 331.2 bp, 330.2 bp, and 339.6 bp) that are not the predominant diagnostic ALH amplicons in CD and UC. Thus, the dysbiosis in Pouchitis seems to be very different from CD and UC and may involve different pathology. Furthermore, the actual components of the community in the disease state vary from individual to individual. For example, the ALH amplicons at 309.2 bp, 310.0 bp, 310.9 by are major components in one patient but are only minor components of others. Importantly, the Normal Pouch patients have abnormal microflora content in the mucosal biofilm and these patients may be continuously in a semi-disease state.

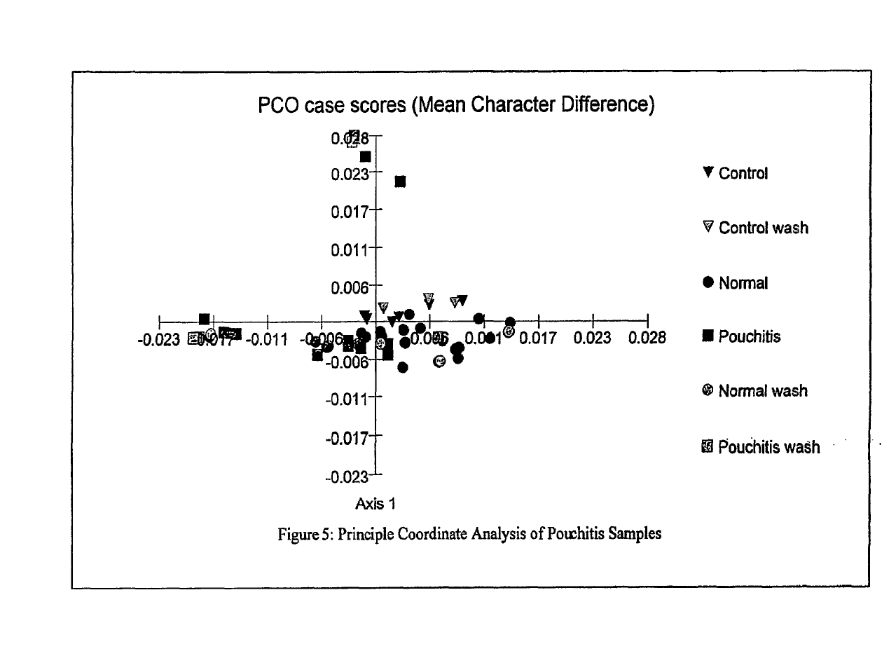

When the mean character differences for ALH profiles from NP and AP samples were examined using Principle Coordinate Analysis (PCO), a general clustering pattern can be seen for NP in the center of the graph that is distinct from three clusters of AP samples (FIG. 5). Interestingly, each of these AP clusters are from separate patient confirming that patients with Pouchitis exhibit much more variation in the microflora in the mucosa]. biofilm. It should be rioted that the separate cluster found on the Y axis above the cluster of Normal Pouch is the patient that displayed the distinct ALH amplicons at 309.2 bp, 310.0 bp, 310.9 bp. The extent of activity of the disease may be reflected in the extent of dysbiosis depicted in the PCO plot. Furthermore, the pattern is consistent whether the samples have been washed or not washed in saline as is the case in the CD and UC samples.

We have cloned and sequenced pooled ALH amplicons from the NP and AP mucosal samples and these sequence data were used to identify the bacterial species associated with each disease state. Table 1 summarizes the key bacterial groups based on the RDP classification scheme that occur at a frequency of greater than 5% of the microfloral community. The data supports the ALH profiles in that the microflora found on the mucosal surface of both AP and NP tissue are different from that found in healthy individuals and these do not reflect the microflora found in Normal lumen as found in CD and UC. Specifically, members of the Clostridium group (i.e. Clostridium paraputrificum), members of Enterics (i.e. E. coli and Shigella sp), and members of the Streptococcus group (i.e. Streptococcus brevis) are found associated with the mucosa of NP patients. On the other hand, the microflora associated with the mucosa in AP patients was very diverse and differed from the NP patients. Specifically, we observed that members of the Enterics (i.e. E. coli and Shigella sp.) and Fusobacterium group (i.e. Fusobacterium varium) was associated with the mucosa in the AP patients and that there was a dramatic loss of members of the Streptococci group (i.e. Streptococcus brevis). Furthermore, a different Ruminococcus species (Ruminococcus obeum) was associated with AP patients but it is not clear that this strain difference would contribute to the pathology. In summary, it looks like both NP and AP patients have dysbiosis compared to the normal controls and that there is a dramatic loss of Streptococci in AP patients. Furthermore, there seems to be patient specific (see FIG. 5) ALE fingerprints suggesting significant variation in the microflora between patients.

### Correlation of ALH Amplicons and Microflora:

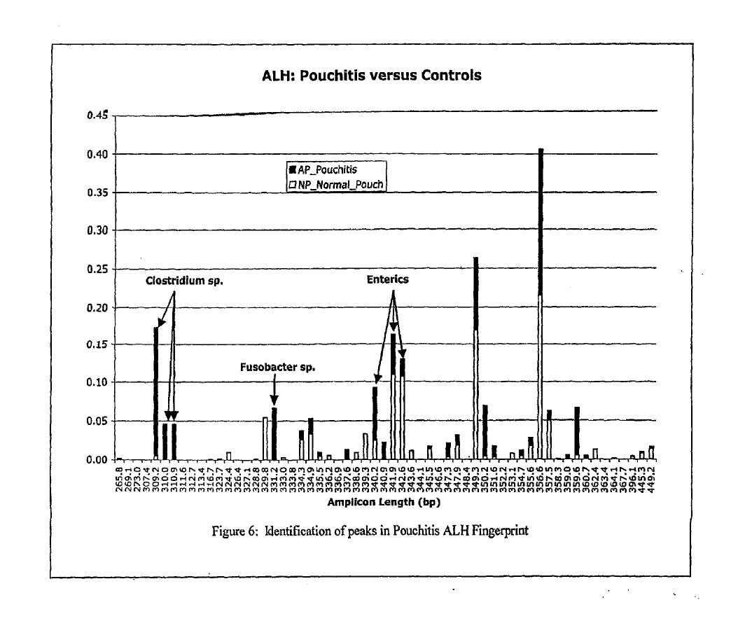

We have correlated the experimentally determinedALH amplicon size of clones with the identifications obtained from sequencing these. For example, we have labeled the main amplicons in the histogram Pouchitis and Normal Pouch ALH fingerprints in FIG. 6. We then use this information to correlate what bacterial species are in the diagnostic peaks of the ALH profiles.

The entire disclosure of all applications, patents and publications, cited herein and of U.S. Provisional Application No. 60/623,771, filed Nov. 1, 2004 and U.S. Provisional Application No. 60/646,592, filed Jan. 26, 2005, are hereby incorporated by reference in their entirety.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

TABLE I
Increase or Decrease of ALH Fingerprint amplicons greater than 5% in Crohns mucosa versus Control Mucosa

| | Amplicon Size (bp) | | | | | | | | | | | | | |
|---|---|---|---|---|---|---|---|---|---|---|---|---|---|---|
| | 333.0 | 334.3 | 336.2 | 337.6 | 338.6 | 340.2 | 341.9 | 342.6 | 343.6 | 347.3 | 347.9 | 349.3 | 351.6 | 358.3 |
| Control Mucosa | | | 2% | 1% | | 6% | 6% | 12% | 1% | 4% | 11% | 21% | 16% | 5% |
| Control Mucosa | 20% | 7% | 2% | 3% | 2% | | 3% | 5% | 6% | | 1% | 31% | 1% | |
| Increased in | 333.0 | 334.3 | | | 338.6 | | | | 343.6 | | | | | |
| Crohns mucosa | | | | | | | | | | | | | | |
| Decreased in | | | | | | | | | | | | | | |
| Crohns mucosa | | | | | | | | | | | | | | |

TABLE 2
Increase or Decrease of ALH Fingerprint amplicons greater than 5% in
Ulcerative Colitis mucosa versus Control Mucosa

| | Amplicon Size (bp) | | | | | | | | |
|---|---|---|---|---|---|---|---|---|---|
| | 333.0 | 334.3 | 340.2 | 341.9 | 342.6 | 347.9 | 349.3 | 351.6 | 358.3 |
| Control Mucosa | | | 6% | 6% | 12% | 11% | 21% | 16% | 5% |
| UC Mucosa | 39% | 13% | 9% | 3% | 7% | | 7% | | |
| Increased in | 333.0 | 334.3 | 340.2 | | | | | | |
| UC mucosa | | | | | | | | | |
| Decreased in | | | | 341.9 | 342.6 | 347.9 | 349.3 | 351.6 | 358.3 |
| UC mucosa | | | | | | | | | |

TABLE 3
Bacterial Species Associated With Pouchitis

| | Example of | Control | Control | Normal | Pouchtis |
|---|---|---|---|---|---|
| RDPGROUP | genus species | Mucosa | Lumen | Pouch | Mucosa |
| BACTEROIDES_GROUP(2.15.1.2.8) | Bacteroides vulgatus | 0.38 | | | 0.28 |
| BACTEROIDES_GROUP(2.15.1.2.7) | Bacteroides distasonis | 0.08 | | | |
| PROPIONIBACTERIUM_GROUP(2.30.1.10.1) | Propionibacterium acnes | 0.06 | | | |
| CLOSTRIDIUM_COCCOIDES_GROUP (2.30.4.1.4) | Ruminococcus gnavus | 0.09 | | 0.13 | 0.08 |
| CLOSTRIDIUM_COCCOIDES_GROUP(2.30.4.1.1) | Ruminococcus obeum | | | | 0.14 |
| PSEUDOMONAS_AND_RELATIVES(2.28.3.13.1.6) | Acinetobacter junii | | 0.10 | | |
| PSEUDOMONAS_AND_RELATIVES(2.28.3.13.1.1) | Moraxella osloensis | | 0.41 | | |
| ACIDOVORAX_GROUP(2.28.2.9.4.14) | Comamonas sp | | 0.14 | | |
| ACIDOVORAX_GROUP(2.28.2.9.4.1) | Comamonas terrigena | | 0.15 | | |
| CYTOPHAG_ [symbol] _GROUP(2.15.1.3.6) | Chryseobacterium balustinum | | | 0.08 | |
| ENTEROCOCCUS_GROUP (2.30.7.20) | Enterococcus hirae | | | | |
| ENTERICS_AND_RELATIVES (2.28.3.27.2) | E. coli/Shigella sp | | | 0.18 | 0.11 |
| STREPTOCOCCI_GROUP (2.30.7.21.6) | Streptococcus bovis | | | 0.34 | |
| FUSOBACTERIA_GROUP (2.29.5) | Fusobacterium varium | | | | 0.12 |

[symbol]

indicates data missing or illegible when filed

TABLE 4
Increase or Decrease of ALH Fingerprint amplicons greater
than 5% in Pouchitis mucosa versus Normal Pouch

| | Amplicon Size (bp) | | | | | | | | | | | | |
|---|---|---|---|---|---|---|---|---|---|---|---|---|---|
| | 309.2 | 310.0 | 310.9 | 329.8 | 331.2 | 340.2 | 341.9 | 342.6 | 349.3 | 350.2 | 356.6 | 357.5 | 359.6 |
| Normal Pouch | 1% | | | 5% | | 2% | 11% | 11% | 17% | | 22% | 5% | 1% |
| Pouchitis | 17% | 5% | 5% | | 7% | 7% | 5% | 2% | 9% | 6% | 19% | 1% | 6% |
| Increased in | 309.2 | 310.0 | 310.9 | | 391.2 | 240.2 | | | | 350.2 | 356.6 | | 359.6 |
| Pouchitis | | | | | | | | | | | | | |
| Decreased in | | | | 329.8 | | | 341.9 | 342.6 | 349.3 | | | 357.5 | |
| Pouchitis | | | | | | | | | | | | | |

TABLE 5
Bacterial Species Associated with Crohns and Ulcerative Colitis

| | | Control | Control | Crohns | Crohns | UC | UC |
|---|---|---|---|---|---|---|---|
| RDP GROUP | Example of Genus species | Mucosa | Lumen | Mucosa | Lumen | Mucosa | Lumen |
| BACTEROIDES_GROUP (2.15.1.2.8) | Bacteroides vulgatus | 0.38 | | | 0.19 | | 0.07 |
| BACTEROIDES_GROUP (2.15.1.2.7) | Bacteroides distasonis | 0.08 | | | | | |
| PROPIONIBACTERIUM_GROUP (2.30.1.10.1) | Propionibacterium acnes | 0.06 | | | | | |
| CLOSTRIDIUM_COCCOIDES_GROUP (2.30.4.1.4) | Ruminococcus gnavus | 0.09 | | | | | |
| CLOSTRIDIUM_COCCOIDES_GROUP (2.30.4.1.1) | Ruminococcus obeum | | | | | | |
| PSEUDOMONAS_AND_RELATIVES (2.28.3.13.1.6) | Acinetobacter Junii | | 0.10 | | | | |
| PSEUDOMONAS_AND_RELATIVES (2.28.3.13.1.1) | Moraxella osloeasis | | 0.41 | 0.29 | 0.15 | 0.10 | 0.30 |
| ACIDOVORAX_GROUP (2.28.2.9.4.14) | Comamonas sp | | 0.14 | 0.13 | 0.12 | 0.12 | 0.14 |
| ACIDOVORAX_GROUP (2.28.2.9.4.1) | Comamonas terrigena | | 0.15 | 0.29 | 0.23 | 0.39 | 0.19 |
| CYTOPHAGA_GROUP_I (2.15.1.3.6) | Chryseobacterium balustinum | | | 0.09 | 0.08 | | 0.11 |
| CLOSTRIDIUM_BOTULINUM_GROUP (2.30.9.2.11.4) | Clostridium paraputrificum | | | | | 0.06 | |
| ENTEROCOCCUS_GROUP (2.30.7.20) | Enterococcus hirae | | | | | 0.06 | |
| ENTERICS_AND_RELATIVES (2.28.3.27.2) | E. coli/Shigella sp | | | | | | |
| STREPTOCOCCI_GROUP (2.30.7.21.6) | Streptococcus bovis | | | | | | |
| FUSOBACTERIA_GROUP (2.29.5) | Fusobacterium varium | | | | | | |

TABLE 6

| RDP REFERENCE | SEQ ID NO | GENUS SPECIES |
|---|---|---|
| 2.28.3.13.1.6 | 14 | Acinetobacter junii |
| 2.15.1.2.7 | 1 | Bacteroides distasonis |
| 2.15.1.2.8 | 2 | Bacteroides fragilis |
| 2.15.1.2.8 | 3 | Bacteroides ovatus |
| 2.15.1.2.8 | 4 | Bacteroides vulgatus |
| 2.15.1.3.6 | 17 | Bergeyella zoohelcum |
| 2.15.1.3.6 | 18 | Chryseobacterium balustinum |
| | | str. SBR1044 |
| 2.15.1.3.6 | 19 | Chryseobacterium balustinum |
| | | str. SBR2024 |
| 2.28.2.9.4.1 | 16 | Comamonas terrigena |
| 2.28.2.9.4.14 | 15 | Comamonas sp. |
| 2.28.3.13.1.1 | 11 | Moraxella cuniculi |
| 2.28.3.13.1.1 | 12 | Moraxella lacunata |
| 2.28.3.13.1.1 | 13 | Moraxella osloensis |
| 2.28.3.27.2 | 25 | Escherichia coli |
| 2.28.3.27.2 | 26 | Salmonella bovis |
| 2.28.3.27.2 | 27 | Shigella boydii |
| 2.28.3.27.2 | 28 | Shigella dysenteriae |
| 2.28.3.27.2 | 29 | Shigella flexneri |
| 2.29.5 | 34 | Fusobacterium alocis |
| 2.29.5 | 35 | Fusobacterium nucleatum |
| 2.29.5 | 36 | Fusobacterium varium |
| 2.30.1.10.1 | 5 | Propionibacterium acnes |
| 2.30.4.1.1 | 10 | Clostridium sp. |
| 2.30.4.1.4 | 6 | Clostridium nexile |
| 2.30.4.1.4 | 7 | Eubacterium formicigenerans |
| 2.30.4.1.4 | 8 | Ruminococcus gnavus |
| 2.30.4.1.4 | 9 | Ruminococcus torques |
| 2.30.7.20 | 21 | Enterococcus cecorum |
| 2.30.7.20 | 22 | Enterococcus columbae |
| 2.30.7.20 | 23 | Enterococcus hirae |
| 2.30.7.20 | 24 | Tetragonococcus halophilus |
| 2.30.7.21.6 | 30 | Streptococcus bovis |
| 2.30.7.21.6 | 31 | Streptococcus infantarius |
| 2.30.7.21.6 | 32 | Streptococcus salivarius |
| 2.30.7.21.6 | 33 | Streptococcus thermophilus |
| 2.30.9.2.11.4 | 20 | Clostridium paraputrificum |

What is claimed is:

1. A method for diagnosing an Inflammatory Bowel Disease in a patient, comprising:
a) obtaining a bacterial rRNA sample isolated from a digestive tract sample of the patient;
b) performing a polymerase chain reaction upon selected variable regions of 16S rRNA from said bacterial rRNA sample, by utilizing a forward and reverse nucleotide primer effective for amplifying bacterial species having SEQ ID NOS:1-36;
c) sequencing the reaction products from said polymerase chain reaction;
d) identifying the bacterial species represented by the sequenced reaction products, wherein bacterial species having SEQ ID NOS:1-36 are present;
e) constructing a bacterial rRNA gene profile comprising the relative abundance for the identified bacterial species having SEQ ID NOS:1-36, by:
i. calculating the total abundance of species having SEQ ID NOS:1-36, and then
ii. computing the relative percentage of said total abundance that is attributable to each individual bacterial species of SEQ ID NOS:1-36;
f) classifying the bacterial rRNA gene profile by computer-implemented cluster analysis to create a cluster pattern in multidimensional space; and
g) diagnosing the patient as having an Inflammatory Bowel Disease, selected from the group consisting of: Crohn's Disease, Ulcerative Colitis, or Pouchitis, by analyzing the patient's classified bacterial rRNA gene profile cluster pattern;
wherein a bacterial rRNA gene profile cluster pattern, indicative of each of Crohn's Disease, Ulcerative Colitis, or Pouchitis, is distinguishable from the other, in multidimensional space.

2. The method of claim 1, wherein the patient is undergoing treatment for an Inflammatory Bowel Disease.

3. The method of claim 1, wherein the digestive tract sample is a stool sample, colonic wash sample, lumen sample, gastric mucosa sample, saliva sample, or intestinal mucosa sample.

4. The method of claim 1, wherein the digestive tract sample is an intestinal mucosa sample.

5. The method of claim 1, wherein the digestive tract sample is a colon sample.

6. The method of claim 1, wherein the clustering is supervised.

7. The method of claim 1, wherein the clustering is unsupervised.

8. The method of claim 1, wherein clustering is done by Principal Components Analysis (PCA), Principal Coordinate Analysis (PCO), Canonical Correspondence Analysis (CCA), C4.5, Support Vector Machines (SVM), hierarchal classification, Unweighted Pair Group Method using Arithmetic Averages (UPGMA), or K-means.


SEQUENCE LISTING (38 sequences)

SEQ ID NO: 1 - RNA, 1489 nt, Bacteroides distasonis
  modified_base (39)..(39): a, c, g, u, unknown or other
  misc_feature (39)..(39): n is a, c, g, or u
  modified_base (99)..(99): a, c, g, u, unknown or other
  misc_feature (99)..(99): n is a, c, g, or u
  modified_base (101)..(101): a, c, g, u, unknown or other
  misc_feature (101)..(101): n is a, c, g, or u
  modified_base (108)..(108): a, c, g, u, unknown or other
  misc_feature (108)..(108): n is a, c, g, or u
  modified_base (235)..(235): a, c, g, u, unknown or other
  misc_feature (235)..(235): n is a, c, g, or u
  modified_base (482)..(482): a, c, g, u, unknown or other
  misc_feature (482)..(482): n is a, c, g, or u
  modified_base (656)..(656): a, c, g, u, unknown or other
  misc_feature (656)..(656): n is a, c, g, or u
  modified_base (932)..(932): a, c, g, u, unknown or other
  misc_feature (932)..(932): n is a, c, g, or u
  modified_base (936)..(936): a, c, g, u, unknown or other
  misc_feature (936)..(936): n is a, c, g, or u
  modified_base (1160)..(1160): a, c, g, u, unknown or other
  misc_feature (1160)..(1160): n is a, c, g, or u
caauuuaaacaacgaagaguuugauccuggcucaggaunaacgcuagcgacaggcuuaacacaugcaagucgaggggcacgcgcgrguagcaauaccgngngcuggcnaccggcgcacgggugaguaacgcguaugcaacuugccuaucagagggggauaacccggcgaaagucggacuaauaccgcaugaagcagggaucccgcaugggaauauuugcuaaagauucaucgcunauagauaggcaugcguuccauuaggcaguuggcgggguaacggcccaccaaaccgacgauggauagggguucugagaggaaggucccccacauugguacugagacacggaccaaacuccuacgggaggcagcagugaggaauauuggucaauggccgagaggcugaaccagccaagucgcgugagggaugaagguucuauggaucguaaaccucuuuuauaagggaauaaagugcgggacguguccnguuuuguauguaccuuaugaauaaggaucggcuaacuccgugccagcagccgcgguaauacggaggauccgagcguuauccggauuuauuggguuuaaagggugcguaggcggccuuuuaagucagcggugaaagucuguggcucaaccauagaauugccguugaaacuggggngcuugaguauguuugaggcaggcggaaugcgugguguagcggugaaaugcauagauaucacgcagaaccccgauugcgaaggcagccugccaagccauuacugacgcugaugcacgaaagcguggggaucaaacaggauuagauacccugguaguccacgcaguaaacgaugaucacuagcuguuugcgauacacuguaagcggcacagcgaaagcguuaagugauccaccuggggaguacgccggcaacggugaaacucaaaggaauugacgggngccngcacaagcggaggaacaugugguuuaauucgaugauacgcgaggaaccuuacccggguuugaacgcauucggaccgagguggaaacaccuuuucuagcaauagccguuugcgaggugcugcaugguugucgucagcucgugccgugaggugucggcuuaagugccauaacgagcgcaacccuugccacuaguuacuaacagguuaggcugaggacucugguggnacugccagcguaagcugcgaggaaggcggggaugacgucaaaucagcacggcccuuacauccggggcgacacacguguuacaauggcguggacaaagggaggccaccuggcgacagggagcgaauccccaaaccacgucucaguucggaucggagucugcaacccgacuccgugaagcuggauucgcuaguaaucgcgcaucagccauggcgcggugaauacguucccgggccuuguacacaccgcccgucaagccaugggagccggggguaccugaaguccguaaccgaaaggaucggccuaggguaaaacuggugacuggggcuaag

SEQ ID NO: 2 - RNA, 1533 nt, Bacteroides fragilis
uuacaacgaagaguuugauccuggcucaggaugaacgcuagcuacaggcuuaacacaugcaagucgaggggcaucaggaagaaagcuugcuuucuuugcuggcgaccggcgcacgggugaguaacacguauccaaccugcccuuuacucggggauagccuuucgaaagaaagauuaauacccgauagcauaaugauuccgcaugguuucauuauuaaaggauuccgguaaaggauggggaugcguuccauuagguuguuggugagguaacggcucaccaagccuucgauggauagggguucugagaggaaggucccccacauuggaacugagacacgguccaaacuccuacgggaggcagcagugaggaauauuggucaaugggcgcuagccugaaccagccaaguagcgugaaggaugaaggcucuaugggucguaaacuucuuuuauauaagaauaaagugcaguauguauacuguuuuguauguauuauaugaauaaggaucggcuaacuccgugccagcagccgcgguaauacggaggauccgagcguuauccggauuuauuggguuuaaagggagcguagguggacugguaagucaguugugaaaguuugcggcucaaccguaaaauugcagcugauacugucagucuugaguacaguagaggugggcggaauucgugguguagcggugaaaugcuuagauaucacgaagaacuccgauugcgaaggcagcucacuggacugcaacugacacugaugcucgaaaguguggguaucaaacaggauuagauacccugguaguccacacaguaaacgaugaauacucgcuguuugcgauauacaguaagcggccaagcgaaagcauuaaguauuccaccuggggaguacgccggcaacggugaaacucaaaggaauugacgggggcccgcacaagcggaggaacaugugguuuaauucgaugauacgcgaggaaccuuacccgggcuuaaauugcaguggaaugauguggaaacaugucagugagcaaucaccgcugugaaggugcugcaugguugucgucagcucgugccgugaggugucggcuuaagugccauaacgagcgcaacccuuaucuuuaguuacuaacagguuaugcugaggacucuagagagacugccgucguaagaugugaggaagguggggaugacgucaaaucagcacggcccuuacguccggggcuacacacguguuacaaugggggguacagaaggcagcuagcgggugaccguaugcuaaucccaaaauccucucucaguucggaucgaagucugcaacccgacuucgugaagcuggauucgcuaguaaucgcgcaucagccacggcgcggugaauacguucccgggccuuguacacaccgcccgucaagccaugggagccggggguaccugaaguacguaaccgcaaggaucguccuaggguaaaacuggugacuggggcuaagucguaacaagguagccguaccggaaggugcggcuggaacaccuccuuucu

SEQ ID NO: 3 - RNA, 1468 nt, Bacteroides ovatus
  modified_base (204)..(207): a, c, g, u, unknown or other
  misc_feature (204)..(207): n is a, c, g, or u
  modified_base (339)..(339): a, c, g, u, unknown or other
  misc_feature (339)..(339): n is a, c, g, or u
  modified_base (439)..(439): a, c, g, u, unknown or other
  misc_feature (439)..(439): n is a, c, g, or u
  modified_base (530)..(530): a, c, g, u, unknown or other
  misc_feature (530)..(530): n is a, c, g, or u
  modified_base (544)..(544): a, c, g, u, unknown or other
  misc_feature (544)..(544): n is a, c, g, or u
  modified_base (700)..(700): a, c, g, u, unknown or other
  misc_feature (700)..(700): n is a, c, g, or u
  modified_base (739)..(739): a, c, g, u, unknown or other
  misc_feature (739)..(739): n is a, c, g, or u
  modified_base (788)..(788): a, c, g, u, unknown or other
  misc_feature (788)..(788): n is a, c, g, or u
  modified_base (953)..(953): a, c, g, u, unknown or other
  misc_feature (953)..(953): n is a, c, g, or u
  modified_base (962)..(962): a, c, g, u, unknown or other
  misc_feature (962)..(962): n is a, c, g, or u
  modified_base (988)..(988): a, c, g, u, unknown or other
  misc_feature (988)..(988): n is a, c, g, or u
  modified_base (1031)..(1031): a, c, g, u, unknown or other
  misc_feature (1031)..(1031): n is a, c, g, or u
  modified_base (1084)..(1084): a, c, g, u, unknown or other
  misc_feature (1084)..(1084): n is a, c, g, or u
augaagaguuugauccuggcucaggaugaacgcuagcuacaggcuuaacacaugcaagucgaggggcagcauuuuaguuugcuugcaaacugaagauggcgaccggcgcacgggugaguaacacguauccaaccugccgauaacuccgggauagccuuucgaaagaaagauuaauaccggaurgyauaygaacaucgcaugaunnnnuuauuaaagaauuucgguuaucgauggggaugcguuccauuaguuuguuggcgggguaacggcccaccaagacuacgauggauagggguucugagaggaaggucccccacauuggaacugagacacgguccnaacuccuacgggaggcagcagugaggaauauuggucaaugggcgagagccugaaccagccaaguagcgugaaggaugaaggcucuaugggucguaaacuncuuuuauaugggaauaaaguguuccacguguggaauuuuguauguaccauaugaauaaggaucggcuaacuccgugccagcagccgcggunauacggaggauccnagcguuauccggauuuauuggguuuaaagggagcguagguggauuguuaagucaguugugaaaguuugcggcucaaccguaaaauugcaguugaaacuggcagucuugaguacaguagaggugggcggaauucgugguguagcggugaaaugcuungauaucacgaagaacuccgauugcgaaggcagcucacungacuguuacugacacugaugcucgaaaguguggguaucaaacaggauungauacccugguaguccacacaguaaacgaugaauacucgcuguuugcgauauacaguaagcggccaagcgaaagcauuaaguauuccaccuggggaguacgccggcaacggugaaacucaaaggaauugacgggggcccgcacaagcggaggaacaugugguuunauucgaugnuacgcgaggaaccuuacccgggcuunaauugcawcwgaauauauwggaaacwruauagccgyaaggcanuugugaaggugcugcaugguugucgucagcucgugccgugaggugucggcuunagugccauaacgagcgcaacccuuaucuuuaguuacuaacagguuaugcugaggacucuagagagacugccgucguaagaugugaggaagguggggaugacgucaaaucagcacggcccuuacguccggggcuacacacguguuacaaugggggguacagaaggcrgcuaccuggygacaggaugcuaaucccaaaaaccucucucaguucggaucgaagucugcaacccgacuucgugaagcuggauucgcuaguaaucgcgcaucagccauggcgcggugaauacguucccgggccuuguacacaccgcccgucaagccaugaaagccggggguaccugaaguacguaaccgcaaggagcguccuaggguaaaacugguaau

SEQ ID NO: 4 - RNA, 1524 nt, Bacteroides vulgatus
  modified_base (35)..(35): a, c, g, u, unknown or other
  misc_feature (35)..(35): n is a, c, g, or u
  modified_base (97)..(97): a, c, g, u, unknown or other
  misc_feature (97)..(97): n is a, c, g, or u
  modified_base (99)..(99): a, c, g, u, unknown or other
  misc_feature (99)..(99): n is a, c, g, or u
  modified_base (395)..(395): a, c, g, u, unknown or other
  misc_feature (395)..(395): n is a, c, g, or u
  modified_base (481)..(481): a, c, g, u, unknown or other
  misc_feature (481)..(481): n is a, c, g, or u
  modified_base (544)..(544): a, c, g, u, unknown or other
  misc_feature (544)..(544): n is a, c, g, or u
  modified_base (744)..(744): a, c, g, u, unknown or other
  misc_feature (744)..(744): n is a, c, g, or u
  modified_base (932)..(932): a, c, g, u, unknown or other
  misc_feature (932)..(932): n is a, c, g, or u
  modified_base (1204)..(1204): a, c, g, u, unknown or other
  misc_feature (1204)..(1204): n is a, c, g, or u
  modified_base (1255)..(1255): a, c, g, u, unknown or other
  misc_feature (1255)..(1255): n is a, c, g, or u
  modified_base (1499)..(1499): a, c, g, u, unknown or other
  misc_feature (1499)..(1499): n is a, c, g, or u
uauuacaaugaagaguuugauccuggcucaggaunaacgcuagcuacaggcuuaacacaugcaagucgaggggcagcauggucuuagcuugcuaagncnauggcgaccggcgcacgggugaguaacacguauccaaccugccgucuacucuuggacagccuucugaaaggaagauuaauacaagauggcaucaugaguccgcauguucacaugauuaaagguauuccgguagacgauggggaugcguuccauuagauaguaggcgggguaacggcccaccuagucuucgauggauagggguucugagaggaaggucccccacauuggaacugagacacgguccaaacuccuacgggaggcagcagugaggaauauuggucaaugggcgagagccngaaccagccaaguagcgugaaggaugacugcccuauggguuguaaacuucuuuuauaaaggaauaaagucggguauggauacccgnuugcauguacuuuaugaauaaggaucggcuaacuccgugccagcagccgcgguaauacggagnauccgagcguuauccggauuuauuggguuuaaagggagcguagauggauguuuaagucaguugugaaaguuugcggcucaaccguaaaauugcaguugauacuggauaucuugagugcaguugaggcaggcggaauucgugguguagcggugaaaugcuuagauaucacgaagaacuccgauugcgaaggcagccugcunagcugcaacugacauugaggcucgaaaguguggguaucaaacaggauuagauacccugguaguccacacgguaaacgaugaauacucgcuguuugcgauauacugcaagcggccaagcgaaagcguuaaguauuccaccuggggaguacgccggcaacggugaaacucaaaggaauugacgggggccngcacaagcggaggaacaugugguuuaauucgaugauacgcgaggaaccuuacccgggcuuaaauugcagaugaauuacggugaaagccguaagccgcaaggcaucugugaaggugcugcaugguugucgucagcucgugccgugaggugucggcuuaagugccauaacgagcgcaacccuuguugucaguuacuaacagguuaugcugaggacucugacaagacugccaucguaagaugugaggaagguggggaugacgucaaaucagcacngcccuuacguccggggcuacacacguguuacaaugggggguacagagggcngcuaccacgcgaguggaugccaauccccaaaaccucucucaguucggacuggagucugcaacccgacuccacgaagcuggauucgcuaguaaucgcgcaucagccacggcgcggugaauacguucccgggccuuguacacaccgcccgucaagucaugggagccggggguaccugaagugcguaaccgcgaggagcgcccuaggguaaaacuggugacuggggcuaagucguaacaagguagcnguaccggaaggaacaccuccuuucu

SEQ ID NO: 5 - RNA, 1173 nt, Propionibacterium acnes
uuggagaguuugauccuggcucaggacgaacgcuggcggcgugcuuaacacaugcaagucgaacggaaaggcccugcuuuuguggggugcucgaguggcgaacgggugaguaacacgugaguaaccugcccuugacuuugggauaacuucaggaaacuggggcuaauaccggauaggagcuccugcugcaugguggggguuggaaaguuucggcgguuggggauggacucgcggcuuaucagcuuguuggugggguaguggcuuaccaaggcuuugacggguagccggccugagagggugaccggccacauugggacugagauacggcccagacuccuacgggaggcagcaguggggaauauugcacaaugggcggaagccugaugcagcaacgccgcgugcgggaugacggccuucggguuguaaaccgcuuucgccugugacgaagcgugagugacgguaauggguaaagaagcacccgcuaacuacgugccagcagccgcggugauacguagggugccaacguuguccggauuuauugggcguaaagggcucguaggugguugaucgcgucggaaguguaaucuuggggcuuaacccugagcgugcuuucgauacggguugacuugaggaagguaggggagaauggaauuccugguggagcgguggaaugcgcagauaucaggaggaacaccaguggcgaaggcgguucucugggccuuuccugacgcugaggagcgaaagcguggggagcgaacaggcuuagauacccugguaguccacgcuguaaacgguggguacuaggugugggguccauuccacggguuccgugccguagcuaacgcuuuaaguaccccgccuggggaguacggccgcaaggcuaaaacucaaaggaauugacggggccccgcacaagcggcggagcaugcggauuaauucgaugcaacgcguagaaccuuaccuggguuugacauggaucgggagugcucagagaugggugugccucuuuuggggucgguucacagguggugcauggcugucgucagcucgugucgugagauguuggguuaagucccgcaacgagcgcaacccuuguucucuguugccagcacguuaugguggggacucaguggagaccgccggggucaacucggaggaagguggggaugacgu

SEQ ID NO: 6 - RNA, 1517 nt, Clostridium nexile
  modified_base (184)..(185): a, c, g, u, unknown or other
  misc_feature (184)..(185): n is a, c, g, or u
  modified_base (341)..(341): a, c, g, u, unknown or other
  misc_feature (341)..(341): n is a, c, g, or u
  modified_base (430)..(430): a, c, g, u, unknown or other
  misc_feature (430)..(430): n is a, c, g, or u
gagauuugauccuggcucaggaugaacgcuggccggccgugcuuacacaugcagucgaacgaagcgcuuaaacuggauuucuucggauugaaguuuuugcugacugaguggcggacgggugaguaacgcguggguaaccugccucauacagggggauaacaguuagaaaugacugcuaauaccnnauaagcgcacagugcugcauggcacaguguaaaaacuccggugguaugagauggacccgcgucugauuagcuaguuggugggguaacggccuaccaaggcgacgaucaguagccggccugagagggugaacggccacauugggacugagacacggnccaaacuccuacgggaggcagcaguggggaauauugcacaaugggggaaacccugaugcagcgacgccgcgugagcgaagaaguauuungguauguaaagcucuaucagcagggaagaaaaugacgguaccugacuaagaagcaccggcuaaauacgugccagcagccgcgguaauacguaggugcaagcguuauccggauuuacuggguguaaagggagcguagacgguuguguaagucugaugugaaagcccggggcucaaccccggacugcauuggaaacuauguaacuagagugucggagagguaagcggaauuccuaguguagcggugaaaugcguagauauuaggaggaacaccaguggcgaaggcggcuuacuggacgaucacugacguugaggcucgaaagcguggggagcaaacaggauuagauacccugguaguccacgccguaaacgaugacuacuaggugucggggagcaaagcucuucggugccgcagcaaacgcaauaaguaguccaccuggggaguacguucgcaagaaugaaacucaaaggaauugacggggacccgcacaagcguggagcaugugguuuaauucgagcaacgcgaagaccuuaccuggucuugacaucccggugaccgguccaguaaugggaccuuuccuucgggacacggugacagguggugcaugguugucgucagcucgugucgugagauguuggguuaagucccgcaacgagcgcaaccccuaucuucaguagccagcauuuaaggugggcacucuggagagacugccagggauaaccuggaggaagguggggaugacgucaaaucaucaugccccuuaugaccagggcuacacacgugcuacaauggcguaaacaaagggaagcgaaccugugaggggaagcaaaucucaaaaauaacgucucaguucggauuguagucugcaacucgacuacaugaagcuggaaucgcuaguaaucgcgaaucagcaugucgcggugaauacguucccgggucuuguacacaccgcccgucacaccaugggagucaguaacgcccgaagucagugacccaaccguaaggagggagcugccgaaggugggaccgauaacuggggugaagucguaacaagguagccguaucggaaggugcggcuggaucaccucc

SEQ ID NO: 7 - RNA, 1480 nt, Eubacterium formicigenerans
  modified_base (1)..(1): a, c, g, u, unknown or other
  misc_feature (1)..(1): n is a, c, g, or u
  modified_base (124)..(126): a, c, g, u, unknown or other
  misc_feature (124)..(126): n is a, c, g, or u
  modified_base (221)..(223): a, c, g, u, unknown or other
  misc_feature (221)..(223): n is a, c, g, or u
  modified_base (288)..(288): a, c, g, u, unknown or other
  misc_feature (288)..(288): n is a, c, g, or u
  modified_base (350)..(351): a, c, g, u, unknown or other
  misc_feature (350)..(351): n is a, c, g, or u
  modified_base (518)..(518): a, c, g, u, unknown or other
  misc_feature (518)..(518): n is a, c, g, or u
  modified_base (535)..(536): a, c, g, u, unknown or other
  misc_feature (535)..(536): n is a, c, g, or u
  modified_base (726)..(726): a, c, g, u, unknown or other
  misc_feature (726)..(726): n is a, c, g, or u
  modified_base (919)..(919): a, c, g, u, unknown or other
  misc_feature (919)..(919): n is a, c, g, or u
  modified_base (922)..(922): a, c, g, u, unknown or other
  misc_feature (922)..(922): n is a, c, g, or u
  modified_base (956)..(957): a, c, g, u, unknown or other
  misc_feature (956)..(957): n is a, c, g, or u
  modified_base (1185)..(1186): a, c, g, u, unknown or other
  misc_feature (1185)..(1186): n is a, c, g, or u
nuuaaacgagaguuugauccuggcucaggaugaacgcuggcggcgugcuuaacacaugcaagucgagcgaagcacauaaguuugauucuucggaugaagacuuuugugacugagcggcggacgnnngaguaacgcguggguaaccugccucauacagggggauaacagyuagaaauggcugcuaauaccgcauaagaccacaguacugcaugguacagugnnnaaaacuccggugguaugagauggacccgcgucugauuagguaguuggugagguaacggcccaccnagccgacgaucaguagccgaccugagagggugaccggccacauugggacugagacacggccnngacuccuacgggaggcagcaguggggaauauugcacaaugggcgaaagccugaugcagcgacgccgcgugaaggaugaaguauuucgguauguaaacuucuaucagcagggaagaaaaugacgguaccugacuaagaagccccggcuaacuacgugccagcagccgngguaauacguagggggnnagcguuauccggauuuacuggguguaaagggagcguagacggcugugcaagucugaagugaaaggcaugggcucaaccuguggacugcuuuggaaacugugcagcuagagugucggagagguaaguggaauuccuaguguagcggugaaaugcguagauauuaggaggaacaccaguggcgaaggcggcnuacuggacgaugacugacguugaggcucgaaagcguggggagcaaacaggauuagauacccugguaguccacgccguaaacgaugacugcuaggugucggguagcaaagcuauucggugccgcagcuaacgcaauaagcaguccaccuggggaguacguucgcaagaaugaaacucaaaggaauugacggggnccngcacaagcgguggagcaugugguuuaauucgaannaacgcgaagaaccuuaccugaucuugacaucccgaugaccgcuucguaauggaagyuuuucuucggaacaucggugacagguggugcaugguugucgucagcucgugucgugagauguuggguuaagucccgcaacgagcgcaacccuuaucuucaguagccagcauuuaggaugggcacucuggagagacugccagggauaaccuggaggaagguggggaugacgunnaaucaucaugccccuuaugaccagggcuacacacgugcuacaauggcguaaacagagggaggcagagccgcgaggccgagcaaaucucaaaaauaacgucucaguucggauuguagucugcaacucgacuacaugaagcuggaaucgcuaguaaucgcagaucagaaugcugcggugaauacguucccgggucuuguacacaccgcccgucacaccaugggagucaguaacgcccgaagucagugacccaaccgaaaggagggagcugccgaaggugggaccgauaacuggggu

SEQ ID NO: 8 - DNA, 1423 bp, Ruminococcus gnavus
  modified_base (1148)..(1148): a, c, g, t, unknown or other
  misc_feature (1148)..(1148): n is a, c, g, or t
  modified_base (1188)..(1188): a, c, g, t, unknown or other
  misc_feature (1188)..(1188): n is a, c, g, or t
  modified_base (1257)..(1257): a, c, g, t, unknown or other
  misc_feature (1257)..(1257): n is a, c, g, or t
cctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggaaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactvccagggataacctggnggaaggtggggatgacgtcaaatcatcatgccccttatgnccagggctacacacgtgctacaatggcgtaaacaaagggaagcgagcccgcgagggggagcaaatcccnaaaataacgtctcagttcggattgtagtctgcaactcgactacatgaagctggaatcgctagtaatcgcgaatcagaatgtcgcggtgaatacgttcccgggtcttgtacacaccscccgtcacaccatgggagtmagtaacgcccgaagtcagtgacccaaccgc

SEQ ID NO: 9 - DNA, 1418 bp, Ruminococcus torques
  modified_base (724)..(724): a, c, g, t, unknown or other
  misc_feature (724)..(724): n is a, c, g, or t
  modified_base (771)..(771): a, c, g, t, unknown or other
  misc_feature (771)..(771): n is a, c, g, or t
  modified_base (776)..(776): a, c, g, t, unknown or other
  misc_feature (776)..(776): n is a, c, g, or t
  modified_base (807)..(807): a, c, g, t, unknown or other
  misc_feature (807)..(807): n is a, c, g, or t
ctcaggatgaacgctggcggcgtgcctaacacatgcaagtcgagcgaagcactttgcttagattcttcggatgaagaggattgtgactgagcggcggacgggtgagtaacgcgtgggtaacctgcctcatacagggggataacagttagaaatgactgctaataccgcataagaccacagcaccgcatggtgcgggggtaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggatgggcaagtctgatgtgaaaacccggggctcaaccccgggactgcattggaaactgttcatctagagtgctggagaggtaagtggaattcctagtgtagcggtgaaatgcgtagatattaggaggaacaccagtggcgaaggcggcttactggacagtaactgacgtngaggctcgaaagcgtggggagcacacaggattagataccctggtagnccacnccgtaaacgatgactactaggtgtcgggtgncaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccgctgaccggacggtaatgcgtccttcccttcggggcagcggagacaggtggtgcatggttgtcgtcagctcgtgtcgtgagatgttgggttaagtcccgcaacgagcgcaacccctatctttagtagccagcggccaggccgggcactctagagagactgccggggataacccggaggaaggtggggatgacgtcaaatcatcatgccccttatgagcagggctacacacgtgctacaatggcgtaaacaaagggaagcgagaccgcgaggtggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 10 - RNA, 1458 nt, Clostridium sp.
gaugaacgcuggcggcgugcuuaacacaugcaagucgagcgaagcgauucuaawgaaguuuucggayggaauuuraauugacugagcggcggacgggugaguaacgcguggguaaccugccucauacagggggauaacaguuggaaacggcugcuaauaccgcauaagcacacagugccgcaugguacggugugaaaaacuccggugguaugagauggacccgcgucugauuagguaguuggugagguaacggcccaccaagccgacgaucaguagccgaccugagagggugaccggccacauugggacugagacacggcccaaacuccuacgggaggcagcaguggggaauauuggacaaugggggaaacccugauccagcgacgccgcgugagugaagaaguauuucgguauguaaagcucuaucagcagggaagaaaaugacgguaccugacuaagaagccccggcuaacuacgugccagcagccgcgguaauacguagggggcaagcguuauccggauuuacuggguguaaagggagcguagacggcaaugcaagucuggagugaaagcccggggcucaaccccgggacugcuuuggaaacuguguugcuagagugcaggagagguaaguggaauuccuaguguagcggugaaaugcguagauauuaggaggaacaccaguggcgaaggcggcuuacuggacuguaacugacguugaggcucgaaagcguggggagcaaacaggauuagauacccugguaguccacgccguaaacgaugaauacuagguguuggggagcaaagcucuucggugccgccgcuaacgcaauaaguauuccaccuggggaguacguucgcaagaaugaaacucaaaggaauugacggggacccgcacaagcgguggagcaugugguuuaauucgaagcaacgcgaagaaccuuaccaagucuugacaucggaaugaccgguccguaacggggccuucccuacggggcauuccagacagguggugcaugguugucgucagcucgugucgugagauguuggguuaagucccgcaacgagcgcaacccuuauccuuaguagccagcauguaguggugggcacucuggggagacugccagggauaaccuggaggaagguggggaugacgucaaaucaucaugccccuuaugauuugggcuacacacgugcuacaauggcguaaacaaagggaagcaaaggagcgaucuuaagcaaaccccaaaaauaacgucucaguucggauuguagucugcaacucgacuacaugaagcuggaaucgcuaguaaucgcggaucagaaugccgcggugaauacguucccgggucuuguacacaccgcccgucacaccaugggaguugguaacgcccgaagucagugacccamccsymaggagggagcugccgaaggcgggacurauaacuggggugaaug

SEQ ID NO: 11 - RNA, 728 nt, Moraxella cuniculi
aggcuuaacacaugcaagucgaacgaaguuagggagcuugcuccugauacuuaguggcggacgggugaguaaugcuuaggaaucugccuaguagugggggauaacuauccgaaaggauagcuaauaccgcauacgaccuacgggugaaagggggcguaagcucucgcuauuagaugagccuaagucggauuagcuaguuggugggguaaaggccuaccaaggcgacgaucuguagcuggucugagaggaugaucagccacacugggacugagacacggcccagacuccuacgggaggcagcaguggggaauauuggacaaugggcgaaagccugauccagccaugcccgcgugugugaagaaggccuuuugguuguaaagcacuuuaaguggggaggaaaagcuaauagcuaauaccuauuagcccugacguuacccacagaauaagcaccggcuaacucugugccagcagccgcgguaauacagagggugcaagcguuaaucggaauuacugggcguaaagcgcgcguaggugguuacuuaagucagaugugaaagccccgggcuuaaccugggaacugcaucugauacuggguaacuagaguaggugagagggaaguagaauuccagguguagcggugaaaugcguagagaucuggaggaauaccgauggcgaaggcagcuuccuggcaucauacugacacugaggugcgaaagcguggguagcaa

SEQ ID NO: 12 - RNA, 1519 nt, Moraxella lacunata
agaguuugaucauggcucagauugaacgcuggcggcaggcuuaacacaugcaagucgaacgaugaagucuagcuugcuagacggauuaguggcgaacgggugaguaaugcuuaggaaucugccuauuagugggggauaacguagggaaacuuacgcuaauaccgcauacgucuuacgagagaaagggggcuuuuagcucucgcuaauagaugagccuaagucggauuagcuaguuggugggguaaaggccuaccaaggcgacgaucuguagcuggucugagaggaugaucagccacacugggacugagacacggcccagacuccuacgggaggcagcaguggggaauauuggacaaugggcgaaagccugauccagccaugccgcgugugugaagaaggccuuuugguuguaaagcacuuuaaguggggaggaaaagcuugugguuaauaccuacaagcccugacguuacccacagaauaagcaccggcuaacucugugccagcagccgcgguaauacagagggugcaagcguuaaucggaauuacugggcguaaagcgagcguagguggucauuuaagucagaugugaaagccccgggcuuaaccugggaacugcaucugauacugggugacuagaguaggugagagggaaguagaauuccagguguagcggugaaaugcguagagaucuggaggaauaccgauggcgaaggcagcuuccuggcaucauacugacacugagguucgaaagcguggguagcaaacaggauuagauacccugguaguccacgccguaaacgaugucuaccagucguugggucucuugaagacuuagugacgcaguuaacgcaauaaguagaccgccuggggaguacggccgcaagguuaaaacucaaaugaauugacggggcccgcacaagcgguggagcaugugguuuaauucgaugcaacgcgaagaaccuuaccuggucuugacauagugagaauccugcagagaugcgggagugcuucgggaauucacauacaggugcugcauggcugucgucagcccgugucgugagauguuggguuaagucccgcaacgagcgcaacccuuuuccuuaguuaccagcgauuuaagucgggaacucuaaggauacugccagugacaaacuggaggaaggcgggacgacgucaagucaucauggcccuuacgaccagggcuacacacgugcuacaaugguugguacaaaggguugcuacacagcgaugugaugcuaaucucaaaaagccaaucguaguccggauuggagucugcaacucgacuccaugaagucggaaucgcuaguaaucgcagaucagaaugcugcggugaauacguucccgggccuuguacacaccgcccgucacaccaugggaguugaucucaccagaagugguuagccuaacgcaagagggcgaucaccacgguggggucgaugacuggggugaagucguaacaagguagccguaggggaacugcgguuggaucaccuccuua

SEQ ID NO: 13 - RNA, 1448 nt, Moraxella osloensis
cuggcggcaggcuuaacacaugcaagucgaacgaugacucucuagcuugcuagagaugauuaguggcggacgggugaguaacauuuaggaaucugccuaguagugggggauagcucggggaaacucgaauuaauaccgcauacgaccuacgggugaaagggggcgcaagcucuugcuauuagaugagccuaaaucagauuagcuaguuggugggguaaaggcccaccaaggcgacgaucuguaacuggucugagaggaugaucagucacaccggaacugagacacgguccggacuccuacgggaggcagcaguggggaauauuggacaaugggggcaacccugauccagccaugccgcgugugugaagaaggccuuuugguuguaaagcacuuuaagcagggaggagaggcuaaugguuaauacccauuagauuagacguuaccugcagaauaagcaccggcuaacucugugccagcagccgcgguaauacagagggugcgagcguuaaucggaauuacugggcguaaagcgaguguagguggcucauuaagucacaugugaaauccccgggcuuaaccugggaacugcaugugauacugguggugcuagaauaugugagagggaaguagaauuccagguguagcggugaaaugcguagagaucuggaggaauaccgauggcgaaggcagcuuccuggcauaauauugacacugagauucgaaagcguggguagcaaacaggauuagauacccugguaguccacgccguaaacgaugucuacuagccguugggguccuugagacuuuaguggcgcaguuaacgcgauaaguagaccgccuggggaguacggccgcaagguuaaaacucaaaugaauugacgggggcccgcacaagcgguggagcaugugguuuaauucgaugcaacgcgaagaaccuuaccuggucuugacauagugagaaucucucagagaugagagagugccuucgggaacucacauacaggugcugcauggcugucgucagcucgugucgugagauguuggguuaagucccgcaacgagcgcaacccuuuuccuuauuugccagcggguuaagccgggaacuuuaaggauacugccagugacaaacuggaggaaggcggggacgacgucaagucaucauggcccuuacgaccagggcuacacacgugcuacaaugguagguacagaggguugcuacacagcgaugugaugcuaaucucaaaaagccuaucguaguccggauuggagucugcaacucgacuccaugaagucggaaucgcuaguaaucgcggaucagaaugccgcggugaauacguucccgggccuuguacacaccgcccgucacaccaugggagucuauugcaccagaaguagguagccuaacgaaagagggcgcuuaccacgguguggucgaugacuggggugaa

SEQ ID NO: 14 - RNA, 452 nt, Acinetobacter junii
caugcaagucgagcggagaugaggugcuugcaccuuaucuuagcggcggacgggugaguaaugcuuaggaaucugccuauuagugggggacaacauuccgaaaggaaugcuaauaccgcauacguccuacgggagaaagcaggggaucuucggaccuugcgcuaauagaugagccuaagucggauuagcuaguuggugggguaaaggccuaccaaggcgacgaucuguagcgggucugagaggaugauccgccacacugggacugagacacggcccagacuccuacgggaggcagcaguggggaauauuggacaauggggggaacccugauccagccaugccgcgugugugaagaaggccuuaugguuguaaagcacuuuaagcgaggaggaggcuacugagacuaauacucuuggauaguggacguuacucgcagaauaagcaccggcuaa

SEQ ID NO: 15 - RNA, 1488 nt, Comamonas sp.
auugaacgcuggcggcaugccuuacacaugcaagucgaacgguaacaggucuucggaugcugacgaguggcgaacgggugaguaauacaucggaacgugcccgauygugggggauaacgaggcgaaagcuuugcuaauaccgcauacgaucuacggaugaaagcgggggaucuucggaccucgcgcggacggagcggccgauggcagauuagguaguuggugggauaaaagcuuaccaagccgacgaucuguagcuggucugagaggaugaucagccacacugggacugagacacggcccagacuccuacgggaggcagcaguggggaauuuuggacaaugggggaaacccugauccagccaugccgcgugcaggaugaaggccuucggguuguaaacugcuuuuguacggaacgaaaaggucucuucuaauaaagggggcccaugacgguaccguaagaauaagcaccggcuaacuacgugccagcagccgcgguaauacguagggugcaagcguuaaucggaauuacugggcguaaagcgugcgcaggcgguuauguaagacagaugugaaauccccgggcucaaccugggaacugcauuugugacugcauggcuugagugcggcagagggggauggaauuccgcguguagcagugaaaugcguagauaugcggaggaacaccgauggcgaaggcaauccccugggccugcacugacgcucaugcacgaaagcguggggagcaaacaggauuagauacccugguaguccacgcccuaaacgaugucaacugguuguugggaauuuguuuucucaguaacgaagcuaacgcgugaaguugaccgccuggggaguacggccgcaagguugaaacucaaaggaauugacggggacccgcacaagcgguggaugaugugguuuaauucgaugcaacgcgaaaaaccuuacccaccuuugacauggcaggaaguccacagagaugaggaugugcucgaaagagaaccugcacacaggugcugcauggcugucgucagcucgugucgugagauguuggguuaagucccgcaacgagcgcaacccuugucauuaguugcuacauuuaguugggcacucuaaugagacugccggugacaaaccggaggaagguggggaugacgucaaguccucauggcccuuauagguggggcuacacacgucauacaauggcugguacaaaggguugccaacccgcgagggggagcuaaucccauaaagccagucguaguccggaucgcagucugcaacucgacugcgugaagucggaaucgcuaguaaucguggaucagaaugucacggugaauacguucccgggucuuguacacaccgcccgucacaccaugggagcgggucucgccagaaguagguagccuaaccgcaaggagggcgcuuaccacggcgggguucgugacuggggugaagucguaacaagguagccguaucggaaggugc

SEQ ID NO: 16 - RNA, 1520 nt, Comamonas terrigena
  modified_base (623)..(623): a, c, g, u, unknown or other
  misc_feature (623)..(623): n is a, c, g, or u
  modified_base (630)..(630): a, c, g, u, unknown or other
  misc_feature (630)..(630): n is a, c, g, or u
aguuugauccuggcucagauugaacgcuggcggcaugcuuuacacaugcaagucgaacggcagcacggacuucggucugguggcgaguggcgaacgggugaguaauacaucggaacgugcccaguugugggggauaacuacucgaaagaguagcuaauaccgcaugaguacugagguugaaagcaggggaucgcaagaccuugcgcaacuggagcggccgauggcagauuagguaguuggugggauaaaagcuuaccaagccgacgaucuguagcuggucugagaggacgaccagccacacugggacugagacacggcccagacuccuacgggaggcagcaguggggaauuuuggacaaugggcgaaagccugauccagcaaugccgcgugcaggaugaaggccuucggguuguaaacugcuuuuguacggaacgaaaagcuucggguuaauaccuggagucaugacgguacccuaagaauaagcaccggcuaacuacgugccagcagccgcgguaauacguagggugcaagcguuaaucggaauuacugggcguaaagcgugcgcaggcggucuuguaagacagaggugaaauccccgggcucaaccugggaacugccuuunugacugnaaggcuggagugcggcagagggggauggaauuccgcguguagcagugaaaugcguagauaugcggaggaacaccgauggcgaaggcaauccccugggccugcacugacgcucaugcacgaaagcguggggagcaaacaggauuagauacccugguaguccacggccuaaacgaugucaacugguuguugggucuuaacugacucaguaacgaagcuaacgcgugaaguugaccgccuggggaguacggccgcaagguugaaacucaaaggaauugacggggacccgcacaagcgguggaugaugugguuuaauuugaugcaacgcgaaaaaccuuacccaccuuugacauguacggaauccuuuagagauagaggagugcucgaaagagagccguaacacaggugcugcauggcugucgucagcucgugucgugagauguuggguuaagucccgcaacgagcgcaacccuugccauuaguugcuacgaaagggcacucuaaugggacuuccggugacaaaccggaggaagguggggaugacgucaaguccucauggcccuuauagguggggcuacacacgucauacaauggcugguacaaaggguugccaacccgcgagggggagcuaaucccauaaagccagucguaguccgguucgcagucugcaacucgacugcgugaagucggaaucgcuaguaaucguggaucagaaugucacggugaauacguucccgggucuuguacacaccgcccgucacaccaugggagcgggucucgccagaaguagguagccuaaccguaaggagggcgcuuaccacggcgggguucgugacuggggugaagucguaacaagguagccguaucggaaggugcggcuggaucaccuccuu

SEQ ID NO: 17 - RNA, 1476 nt, Bergeyella zoohelcum
  modified_base (138)..(138): a, c, g, u, unknown or other
  misc_feature (138)..(138): n is a, c, g, or u
  modified_base (141)..(143): a, c, g, u, unknown or other
  misc_feature (141)..(143): n is a, c, g, or u
  modified_base (183)..(183): a, c, g, u, unknown or other
  misc_feature (183)..(183): n is a, c, g, or u
  modified_base (278)..(278): a, c, g, u, unknown or other
  misc_feature (278)..(278): n is a, c, g, or u
  modified_base (285)..(285): a, c, g, u, unknown or other
  misc_feature (285)..(285): n is a, c, g, or u
  modified_base (317)..(318): a, c, g, u, unknown or other
  misc_feature (317)..(318): n is a, c, g, or u
  modified_base (341)..(342): a, c, g, u, unknown or other
  misc_feature (341)..(342): n is a, c, g, or u
  modified_base (344)..(344): a, c, g, u, unknown or other
  misc_feature (344)..(344): n is a, c, g, or u
  modified_base (528)..(528): a, c, g, u, unknown or other
  misc_feature (528)..(528): n is a, c, g, or u
  modified_base (532)..(533): a, c, g, u, unknown or other
  misc_feature (532)..(533): n is a, c, g, or u
  modified_base (542)..(542): a, c, g, u, unknown or other
  misc_feature (542)..(542): n is a, c, g, or u
  modified_base (547)..(547): a, c, g, u, unknown or other
  misc_feature (547)..(547): n is a, c, g, or u
  modified_base (737)..(737): a, c, g, u, unknown or other
  misc_feature (737)..(737): n is a, c, g, or u
  modified_base (766)..(767): a, c, g, u, unknown or other
  misc_feature (766)..(767): n is a, c, g, or u
  modified_base (965)..(966): a, c, g, u, unknown or other
  misc_feature (965)..(966): n is a, c, g, or u
  modified_base (1022)..(1022): a, c, g, u, unknown or other
  misc_feature (1022)..(1022): n is a, c, g, or u
  modified_base (1085)..(1086): a, c, g, u, unknown or other
  misc_feature (1085)..(1086): n is a, c, g, or u
  modified_base (1096)..(1096): a, c, g, u, unknown or other
  misc_feature (1096)..(1096): n is a, c, g, or u
  modified_base (1205)..(1205): a, c, g, u, unknown or other
  misc_feature (1205)..(1205): n is a, c, g, or u
  modified_base (1288)..(1289): a, c, g, u, unknown or other
  misc_feature (1288)..(1289): n is a, c, g, or u
  modified_base (1384)..(1384): a, c, g, u, unknown or other
  misc_feature (1384)..(1384): n is a, c, g, or u
  modified_base (1406)..(1406): a, c, g, u, unknown or other
  misc_feature (1406)..(1406): n is a, c, g, or u
auacaauggagaguuugauccuggcucaggaugaacgcuagcgggaggccuaacacaugcaagccgagcgggauuuguugguuagcuugcuaacuaacaaugagagcggcguacgggugcguaacacgugugcaaccngcnnnuaucugggggauagccuuucgaaaggaagauuaauacccnauaauauauugauuggcaucaguuaauauugaaagcuccggcggauagagaugggcacgcguaagauuagcuaguuggugagguaacggcucacnaaggcuncgaucuuuagggggccugagagggugaucccnnacacugguacugagacacggacnngncuccuacgggaggcagcagugaggaauauuggacaaugggugagagccugauccagccaucccgcgugaaggacuaaggaccuaugguuuguaaacuucuuuuauacagggauaaaccuacucucgugaggguagcugaagguacuguaugaauaagcaccggcuaacuccgugccagcagccncggnnauacggagngugcnagcguuauccggauuuauuggguuuaaaggguccguaggcgggucgauaagucaguggugaaagccugcagcuuaacuguagaacugccguugauacugucggucuugaguguauuugagguagcuggaaugaguaguguagcggugaaaugcauagauauuacucagaacaccaauugcgaaggcaggnuaccaaguuacaacugacgcugauggacnnaagcguggggagcgaacaggauuagauacccugguaguccacgcuguaaacgaugcuaacucguuuuugggguauuauacuucagagaccaagcgaaagugauaaguuagccaccuggggaguacgaacgcaaguuugaaacucaaaggaauugacgggggcccgcacaagcgguggauuaugugguuuaauucgaunnuacgcgaggaaccuuaccaagacuuaaaugggaauugacagcuguagaaauacggnuuucuucggacaauuuucaaggugcugcaugguugucgucagcucgugccgugagguguuagnnuaaguccugnaacgagcgcaaccccugucacuaguugccaucauuaaguuggggacucuagugagacugccuacgcaaguagagaggaagguggggaugacgucaaaucaucacggccnuuacgucuugggccacacacguaauacaauggccgguacagagggcagcuacacugcgaagugaugcgaaucucgaaagccgnncucaguucggauuggagucugcaacucgacucuaugaagcuggaaucgcuaguaaucgcgcaucagccauggcgcggugaauacguucccgggcnuuguacacaccgcccgucaagncauggaagucugggguaccugaagucggugaccguuaaaggagcugccuaggguaaaacagguaacuagg

SEQ ID NO: 18 - RNA, 395 nt, Chryseobacterium balustinum str. SBR1044
  modified_base (343)..(343): a, c, g, u, unknown or other
  misc_feature (343)..(343): n is a, c, g, or u
uagcgggagggcuaacacaugcaagccgagcgguauuguuucuucggaaaugagagagcggcguacgggugcggaacacgugugcaaccugccuuuaucugggggauagccuuucgaaaggaagauuaauacuccauaacauauugaacggcaucguuugauauugaaagcuccggcggauagagaugggcacgcgcaagauuagcuaguuggugagguaacggcucaccaaggcgaugaucuuuaggggggcugagagggugaucccccacacugguacugagacacggaccagacuccuacgggaggcagcagugaggaauauuggucaaugggugcaagncugaaccagccaucccgcgugaaggacgacugcccuauggguuguaaacuuc

SEQ ID NO: 19 - RNA, 395 nt, Chryseobacterium balustinum str. SBR2024
  modified_base (11)..(11): a, c, g, u, unknown or other
  misc_feature (11)..(11): n is a, c, g, or u
  modified_base (14)..(14): a, c, g, u, unknown or other
  misc_feature (14)..(14): n is a, c, g, or u
  modified_base (17)..(18): a, c, g, u, unknown or other
  misc_feature (17)..(18): n is a, c, g, or u
  modified_base (310)..(310): a, c, g, u, unknown or other
  misc_feature (310)..(310): n is a, c, g, or u
uagcgggaggncunacnnaugcaagccgagcgguauuguuucuucggaaaugagagagcggcguacgggugcggaacacgugugcaaccugccuuuaucugggggauagccuuucgaaaggaagauuaauacuccauaacauauugauuggcaucaauuaauauugaaagcuccggcggauagagaugggcacgcgcaagauuagcuaguuggugagguaacggcucaccaaggcgaugaucuuuaggggggcugagagggugaucccccacacugguacugagacacggaccagacuccuacgggaggnagcagugaggaauauuggucaaugggugcaagccugaaccagccaucccgcgugaaggacgacugcccuauggguuguaaacuuc

SEQ ID NO: 20 - RNA, 1435 nt, Clostridium paraputrificum
cgaacgcuggcggcgugccuaacacaugcaagucgagcgaugaaguuccuucgggaacggauuagcggcggacgggugaguaacacgugggcaaccugccuuauagaggggaauagccuuccgaaaggaagauuaauaccgcauaagauuguagcuucgcaugaaguagcaauuaaaggagcaauccgcuauaagaugggcccgcggcgcauuagcuaguuggugagguaacggcucaccaaggcgacgaugcguagccgaccugagagggugaucggccacauugggacugagacacggcccagacuccuacgggaggcagcaguggggaauauugcacaaugggggaaacccugaugcagcaacgccgcgugagugaugacggccuucggguuguaaagcucugucuuuggggacgauaaugacgguacccaaggaggaagccacggcuaacuacgugccagcagccgcgguaauacguagguggcaagcguuguccggauuuacugggcguaaagggagcguaggcggauuuuuaagugggaugugaaauacccgggcucaaccugggugcugcauuccaaacuggaaaucuagagugcaggaggggaaaguggaauuccuaguguagcggugaaaugcguagagauuaggaagaacaccaguggcgaaggcgacuuucuggacuguaacugacgcugaggcucgaaagcguggggagcaaacaggauuagauacccugguaguccacgccguaaacgaugaauacuagguguagggguugucaugaccucugugccgccgcuaacgcauuaaguauuccgccuggggaguacggucgcaagauuaaaacucaaaggaauugacgggggcccgcacaaguagcggagcaugugguuuaauucgaagcaacgcgaagaaccuuaccuagacuugacaucuccugaauuaccauguaaugugggaagucccuucggggacaggaagacagguggugcaugguugucgucagcucgugucgugagauguuggguuaagucccgcaacgagcgcaacccuuauuguuaguugcuaccauuuaguugagcacucuagcgagacugcccggguuaaccgggaggaagguggggaugacgucaaaucaucaugccccuuaugucuagggcuacacacgugcuacaauggccgguacaacgagaugcaauaccgugagguggagcaaaacuauaaaaccggucucaguucggauuguaggcugaaacucgccuacaugaagcuggaguuacuaguaaucgcgaaucagaaugucgcggugaauacguucccgggccuuguacacaccgcccgucacaccaugagaguuggcaauacccaaaguuggugaucuaacccguaagggaggaagccaccuaagguagggucagcgauuggggug

SEQ ID NO: 21 - RNA, 1509 nt, Enterococcus cecorum
gacgaacgcuggcggcgugccuaauacaugcaagucgaacgcauuuucuuucaccguagcuugcuacaccggaagaaaaugaguggcgaacgggugaguaacacguggguaaccugcccaucagcgggggauaacacuuggaaacaggugcuaauaccgcauaauuccauuuaccgcaugguagauggaugaaaggcgcuuuugcgucacugauggauggacccgcggugcauuagcuaguuggugggguaacggccuaccaaggcugcgaugcauagccgaccugagagggugaucggccacacugggacugagacacggcccagacuccuacgggaggcagcaguagggaaucuucggcaauggacgcaagucugaccgagcaacgccgcgugagugaagaagguuuucggaucguaaaacucuguuguuagagaagaacaaggaugagaguggaaaguucaucccuugacgguaucuaaccagaaagccacggcuaacuacgugccagcagccgcgguaauacguagguggcaagcguuguccggauuuauugggcguaaagcgagcgcaggcggucuuuuaagucugaugugaaagcccccggcuuaaccggggagggucauuggaaacugggagacuugagugcagaagaggaaagcggaauuccauguguagcggugaaaugcguagauauauggaggaacaccaguggcgaaggcggcuuucuggucuguaacugacgcugaggcucgaaagcguggggagcaaacaggauuagauacccugguaguccacgccguaaacgaugagugcuaaguguuggaggguuuccgcccuucagugcugcagcaaacgcauuaagcacuccgccuggggaguacgaccgcaagguugaaacucaaaggaauugacggggacccgcacaagcgguggagcaugugguuuaauucgaagcaacgcgaagaaccuuaccaggucuugacauccuuugaccauccuagagauaggauuuucccuucggggacaaagugacagguggugcaugguugucgucagcucgugucgugagauguuggguuaagucccgcaacgagcgcaacccuuauuguuaguugccaucauucaguugggcacucuagcgagacugccgcagacaaugcggaggaagguggggaugacgucaaaucaucaugccccuuaugaccugggcuacacacgugcuacaauggagaguacaacgagucgcaaagccgcgaggcuaagccaaucucuuaaagcucuucucaguucggauuguaggcugcaacucgccuacaugaagccggaaucgcuaguaaucgcggaucagcacgccgcggugaauacguucccgggucuuguacacaccgcccgucacaccacgagaguuuguaacacccaaagccggugcgguaaccgcaaggagccagccgucuaaggugggauagaugauuggggugaagucguaacaagguagccguaucggaa

SEQ ID NO: 22 - RNA, 1493 nt, Enterococcus columbae
  modified_base (33)..(33): a, c, g, u, unknown or other
  misc_feature (33)..(33): n is a, c, g, or u
  modified_base (103)..(103): a, c, g, u, unknown or other
  misc_feature (103)..(103): n is a, c, g, or u
  modified_base (158)..(158): a, c, g, u, unknown or other
  misc_feature (158)..(158): n is a, c, g, or u
  modified_base (194)..(196): a, c, g, u, unknown or other
  misc_feature (194)..(196): n is a, c, g, or u
  modified_base (209)..(209): a, c, g, u, unknown or other
  misc_feature (209)..(209): n is a, c, g, or u
  modified_base (230)..(230): a, c, g, u, unknown or other
  misc_feature (230)..(230): n is a, c, g, or u
  modified_base (254)..(254): a, c, g, u, unknown or other
  misc_feature (254)..(254): n is a, c, g, or u
  modified_base (289)..(289): a, c, g, u, unknown or other
  misc_feature (289)..(289): n is a, c, g, or u
  modified_base (316)..(316): a, c, g, u, unknown or other
  misc_feature (316)..(316): n is a, c, g, or u
  modified_base (346)..(347): a, c, g, u, unknown or other
  misc_feature (346)..(347): n is a, c, g, or u
  modified_base (367)..(367): a, c, g, u, unknown or other
  misc_feature (367)..(367): n is a, c, g, or u
  modified_base (430)..(432): a, c, g, u, unknown or other
  misc_feature (430)..(432): n is a, c, g, or u
  modified_base (441)..(441): a, c, g, u, unknown or other
  misc_feature (441)..(441): n is a, c, g, or u
  modified_base (492)..(492): a, c, g, u, unknown or other
  misc_feature (492)..(492): n is a, c, g, or u
  modified_base (555)..(556): a, c, g, u, unknown or other
  misc_feature (555)..(556): n is a, c, g, or u
  modified_base (565)..(565): a, c, g, u, unknown or other
  misc_feature (565)..(565): n is a, c, g, or u
  modified_base (613)..(613): a, c, g, u, unknown or other
  misc_feature (613)..(613): n is a, c, g, or u
  modified_base (639)..(639): a, c, g, u, unknown or other
  misc_feature (639)..(639): n is a, c, g, or u
  modified_base (783)..(783): a, c, g, u, unknown or other
  misc_feature (783)..(783): n is a, c, g, or u
  modified_base (788)..(788): a, c, g, u, unknown or other
  misc_feature (788)..(788): n is a, c, g, or u
  modified_base (792)..(792): a, c, g, u, unknown or other
  misc_feature (792)..(792): n is a, c, g, or u
  modified_base (811)..(811): a, c, g, u, unknown or other
  misc_feature (811)..(811): n is a, c, g, or u
  modified_base (871)..(871): a, c, g, u, unknown or other
  misc_feature (871)..(871): n is a, c, g, or u
  modified_base (898)..(898): a, c, g, u, unknown or other
  misc_feature (898)..(898): n is a, c, g, or u
  modified_base (965)..(965): a, c, g, u, unknown or other
  misc_feature (965)..(965): n is a, c, g, or u
  modified_base (979)..(979): a, c, g, u, unknown or other
  misc_feature (979)..(979): n is a, c, g, or u
  modified_base (1013)..(1013): a, c, g, u, unknown or other
  misc_feature (1013)..(1013): n is a, c, g, or u
  modified_base (1035)..(1037): a, c, g, u, unknown or other
  misc_feature (1035)..(1037): n is a, c, g, or u
  modified_base (1063)..(1063): a, c, g, u, unknown or other
  misc_feature (1063)..(1063): n is a, c, g, or u
  modified_base (1068)..(1068): a, c, g, u, unknown or other
  misc_feature (1068)..(1068): n is a, c, g, or u
  modified_base (1071)..(1071): a, c, g, u, unknown or other
  misc_feature (1071)..(1071): n is a, c, g, or u
  modified_base (1102)..(1102): a, c, g, u, unknown or other
  misc_feature (1102)..(1102): n is a, c, g, or u
  modified_base (1127)..(1128): a, c, g, u, unknown or other
  misc_feature (1127)..(1128): n is a, c, g, or u
  modified_base (1228)..(1228): a, c, g, u, unknown or other
  misc_feature (1228)..(1228): n is a, c, g, or u
  modified_base (1337)..(1337): a, c, g, u, unknown or other
  misc_feature (1337)..(1337): n is a, c, g, or u
  modified_base (1399)..(1399): a, c, g, u, unknown or other
  misc_feature (1399)..(1399): n is a, c, g, or u
  modified_base (1401)..(1401): a, c, g, u, unknown or other
  misc_feature (1401)..(1401): n is a, c, g, or u
  modified_base (1424)..(1424): a, c, g, u, unknown or other
  misc_feature (1424)..(1424): n is a, c, g, or u
  modified_base (1462)..(1462): a, c, g, u, unknown or other
  misc_feature (1462)..(1462): n is a, c, g, or u
ugagaguuugauccuggcucaggacgaacgcungcggcgugccuaauacaugcaagucgaacgcacuuucuuucaccguagcuugcuacaccgaaaguaagunaguggcgaacgggugaguuacacguggguaaccugcccaucagcgggggauaacncuuggaaacaggugcuaauaccgcauaauauuacunnncgcaugagaagunauugaaaggcgcaacugcgunacugauggauggacccgcggugcnuuagcuaguuggugagguaacggccuaccaaggcnacgaugcauagccgaccugagagggunaucggccacacugggacugagacacggccnnaacuccuacgggaggcagcnguagggaaucuucggcaauggacgcaagucugaccgagcaacgccgcgugagugaagaaggunnncggaucgunaaacucuguuguuagagaagaacagggaugagaguggaaaguucaucccungacgguaucuaaccagaaagccacggcuaacuacgugccagcagccgcgguaauacguaggunncaagcguunuccggauuuauugggcguaaagcgagcgcaggcggucuuuuaagucunaugugaaagcccacggcuuaaccgungagggucauuggaaacugggagacuugagugcagaagaggaaagcggaauuccauguguagcggugaaaugcguagauauaugcaggaacaccaguggcgaaggcggcuuucuggucuguaacugacgcugaggcucgaaagcnugggnaggnaacaggauuagauacccunguaguccacgccguaaacgaugagugcuaaguguuggaggguuuccgcccuucagugcuncagcaaacgcauuaagcacuccgccungggaguacgaccgcaagguugaaacucaaaggaauugacggggaccgcacaagcgguggagcaugunguuuaauucgaagnaacgcgaagaaccuuaccaggucuugacauccunugaccauccuagagauaggacnnnccuucggggacaaagugacagguggngcaungungucgucagcucgugucgugagauguugggunaagucccgcaacgagcgcaacccunnuuguuaguugccaucauuuaguugggcacucuagcgagacugccgcagacaaugcggaggaagguggggaugacgucaaaucaucaugccccuuaugacnugggcuacacacgugcuacaauggagaguacaacgaguugcgaagucgugaggcuaagcuaaucucuuaaagcucuucucaguucggauuguaggcugcaacucgccuncaugaagccggaaucgcuaguaaucgcggaucagcacgccgcggugaauacguucccgggununguacacaccgcccgucacaccangagaguuuguaacacccgaagccggugggguaaccgcnaggagccagccgucuaaggugggauagauga

SEQ ID NO: 23 - RNA, 1535 nt, Enterococcus hirae
ccuggcucaggacgaacgcuggcggcgugccuaauacaugcaagucgaacgcuucuuuuuccaccggagcuugcuccaccggaaaaagaggaguggcgaacgggugaguaacacguggguaaccugcccaucagaaggggauaacacuuggaaacaggugcuaauaccguauaacaaucgaaaccgcaugguuuugauuugaaaggcgcuuucgggugucgcugauggauggacccgcggugcauuagcuaguuggugagguaacggcucaccaaggcgacgaugcauagccgaccugagagggugaucggccacauugggacugagacacggcccaaacuccuacgggaggcagcaguagggaaucuucggcaauggacgaaagucugaccgagcaacgccgcgugagugaagaagguuuucggaucguaaaacucuguuguuagagaagaacaaggaugagaguaacuguucaucccuugacgguaucuaaccagaaagccacggcuaacuacgugccagcagccgcgguaauacguagguggcaagcguuguccggauuuauugggcguaaagcgagcgcaggcgguuucuuaagucugaugugaaagcccccggcucaaccggggagggucauuggaaacugggagacuugagugcagaagaggagaguggaauuccauguguagcggugaaaugcguagauauauggaggaacaccaguggcgaaggcggcucucuggucuguaacugacgcugaggcucgaaagcguggggagcaaacaggauuagauacccugguaguccacgccguaaacgaugagugcuaaguguuggaggguuuccgcccuucagugcugcagcuaacgcauuaagcacuccgccuggggaguacgaccgcaagguugaaacucaaaggaauugacgggggcccgcacaagcgguggagcaugugguuuaauucgaagcaacgcgaagaaccuuaccaggucuugacauccuuugaccacucuagagauagagcuuccccuucgggggcaaagugacagguggugcaugguugucgucagcucgugucgugagauguuggguuaagucccgcaacgagcgcaacccuuauuguuaguugccaucauuuaguugggcacucuagcaagacugccggugacaaaccggaggaagguggggaugacgucaaaucaucaugccccuuaugaccugggcuacacacgugcuacaaugggaaguacaacgagucgcaaagucgcgaggcuaagcuaaucucuuaaagcuucucucaguucggauuguaggcugcaacucgccuacaugaagccggaaucgcuaguaaucgcggaucagcacgccgcggugaauacguucccgggccuuguacacaccgcccgucacaccacgagaguuuguaacacccgaagucggugagguaaccuuuuggagccagccgccuaaggugggauagaugauuggggugaagucguaacaagguagccguaucggaaggugcggcuggauca

SEQ ID NO: 24 - DNA, 1435 bp, Tetragenococcus halophilus
  modified_base (6)..(6): a, c, g, t, unknown or other
  misc_feature (6)..(6): n is a, c, g, or t
  modified_base (53)..(53): a, c, g, t, unknown or other
  misc_feature (53)..(53): n is a, c, g, or t
  modified_base (253)..(253): a, c, g, t, unknown or other
  misc_feature (253)..(253): n is a, c, g, or t
  modified_base (298)..(298): a, c, g, t, unknown or other
  misc_feature (298)..(298): n is a, c, g, or t
  modified_base (1094)..(1094): a, c, g, t, unknown or other
  misc_feature (1094)..(1094): n is a, c, g, or t
  modified_base (1182)..(1182): a, c, g, t, unknown or other
  misc_feature (1182)..(1182): n is a, c, g, or t
  modified_base (1426)..(1426): a, c, g, t, unknown or other
  misc_feature (1426)..(1426): n is a, c, g, or t
aatacntgcaagtcgaacgctgcttaagaagaaacttcggttttttcttaagnggagtggcggacgggtgagtaacacgtggggaacctatccatcagcgggggataacacttggaaacaggtgctaataccgcatatggctttttttcacctgaaagaaagctcaaaggcgctttacagcgtcactgatggctggtcccgcggtgcattagccagttggtgaggtaacggctcaccaaagcaacgatgcatngccgacctgagagggtgatcggccacactgggactgagacacggnccagactcctacgggaggcagcagtagggaatcttcggcaatggacgcaagtctgaccgagcaacgccgcgtgagtgaagaaggttttcggatcgtaaagctctgttgtcagcaaagaacaggagaaagaggaaatgctttttctatgacggtagctgaccagaaagccacggctaactacgtgccagcagccgcggtaatacgtaggtggcaagcgttgtccggatttattgggcgtaaagcgagcgcaggcggtgatttaagtctgatgtgaaagcccccagctcaactggggagggtcattggaaactggatcacttgagtgcagaagaggagagtggaattccatgtgtagcggtgaaatgcgtagatatatggaggaacaccagtggcgaaggcggctctctggtctgtaactgacgctgaggctcgaaagcgtgggtagcaaacaggattagataccctggtagtccacgccgtaaacgatgagtgctaagtgttggagggtttccgcccttcagtgctgcagttaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccgccctagagatagggtttccccttcgggggcaaagtgacaggtggtgcatggttgtcgtcagctcgtgtcgtgagatgttgggttaagtcccgtaacgagcgcaacccttattgttagttgncagcattgagttgggcactctagcaagactgccggtgacaaaccggaggaaggcggggatgacgtcaaatcatcatgccccttatganctgggctacacacgtgctacaatgggaagtacaacgagcaagccaagccgcaaggcctagcgaatctctgaaagcttctctcagttcggattgcaggctgcaactcgcctgcatgaagccggaatcgctagtaatcgcggatcagcatgccgcggtgaatccgttcccgggccttgtacacaccgcccgtcacaccacgagagtttgtaacacccaaagtcggtgcggcaacccttcggggagncagccgcct

SEQ ID NO: 25 - RNA, 1452 nt, Escherichia coli
  modified_base (491)..(492): a, c, g, u, unknown or other
  misc_feature (491)..(492): n is a, c, g, or u
  modified_base (567)..(567): a, c, g, u, unknown or other
  misc_feature (567)..(567): n is a, c, g, or u
  modified_base (571)..(571): a, c, g, u, unknown or other
  misc_feature (571)..(571): n is a, c, g, or u
  modified_base (852)..(853): a, c, g, u, unknown or other
  misc_feature (852)..(853): n is a, c, g, or u
aguuugaucauggcucagauugaacgcuggcggcaggccuaacacaugcaagucgaacgguaacaggaacgagcuugcugcuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaaaggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaannaccggcuaacuccgugccagcagccgcgguaauacggagggugcaagcguuaaucggaauuacugggcguaaagngcangcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggannuaacgcguuaagucgaccgccuggggaguacggccgcaagguuaaaacucaaaugaauugacgggggccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccuucgggagggcgc

SEQ ID NO: 26 - RNA, 1541 nt, Salmonella bovis
aaauugaagaguuugaucauggcucagauugaacgcuggcggcaggccuaacacaugcaagucgaacgguaacaggaagaagcuugcucgcugcugacgaguggcggacgggugcguaaugucugggaaacugccugauggagggggauaacuacuggaaacgguggcuaaucccgcauaacgucgcaagaccaaagagggggaccuccaggccucuucccaucggaugugcccagaugggauuagcuaguuggugagguaacggcucaccaaggcgacgaucccuagcuggucugagaggaugaccagccacacuggaacugagacacgguccagacuccuacgggaggcagcaguggggaauauugcacagugugcgcaagccugaugcagccaugccgccuguaugaagaaggccuucggguuguaaaguacuuucagcggggaggaagguguugugguuaauaacugcagcaauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucuacuuggagguugugcccuugaggcguggcuuccggagcuaacgcguuaaguagaccgccuggggaguacggccgcaagguuaaaacucaaaugaauugacgggggcccgcacaagcgguggagcaugugguuuaauuccaugcaacucuaagaaccuuaccuggucaugacauccacagaacuuuccagagaugagacugugccuucgggaacugugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucaugccccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccuucgggagggcgcuuaccacuuugugauucaugacuggggugaagucguaacaagguaaccguaggggaaccugcgguuggaucaccuccuua

SEQ ID NO: 27 - RNA, 1488 nt, Shigella boydii
uggcucagauugaacgcuggcggcaggccuaacacaugcaagucgaacgguaacaggaagcagcuugcuguuucgcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuuguuggugggguaacggcucaccaaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuauccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccuucgggagggcgcuuaccacuuugugauucaugacuggggugaagucguaacaaggua

SEQ ID NO: 28 - RNA, 1471 nt, Shigella dysenteriae
  modified_base (1)..(2): a, c, g, u, unknown or other
  misc_feature (1)..(2): n is a, c, g, or u
  modified_base (1470)..(1470): a, c, g, u, unknown or other
  misc_feature (1470)..(1470): n is a, c, g, or u
nnauugaagaguuugaucauggcucagauugaacgcuggcggcaggccuaacacaugcaagucgaacgguaacaggaagcagcuugcugcuuugcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaauggcucaccuaggcgacgaucccuagcuggucugagaggaugaccagccacacuggaacugagacacgguccagacuccuacgggagggagcaguggggaauauugcacaaugggcgcaagccugaugcagccaugccgcguguaugaagaaggcuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaacuucgggagggcgcuuaccacuuunu

SEQ ID NO: 29 - RNA, 1468 nt, Shigella flexneri
  modified_base (1)..(2): a, c, g, u, unknown or other
  misc_feature (1)..(2): n is a, c, g, or u
  modified_base (372)..(372): a, c, g, u, unknown or other
  misc_feature (372)..(372): n is a, c, g, or u
nnauugaagaguuugaucauggcucagauugaacgcuggcggcaggccuaacacaugcaagucgaacgguaacaggaagcagcuugcuguuucgcugacgaguggcggacgggugaguaaugucugggaaacugccugauggagggggauaacuacuggaaacgguagcuaauaccgcauaacgucgcaagaccaaagagggggaccuucgggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcanaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacucacgcucaggugcgaaagcguggggagcaaacaggauuagauacccugguaguccacgcuguaaacgaugucgacuuggagguugugcccuugagguguggcuuccggacguaacgcguuaagucgaccgccuggggaguacggccgcaagguuaaaacucaaaugaauugacgggggccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucacaaagugcgucguaguccggauuggagucugcaacucgacuccaugaagucggaaucgcuaguaaucguggaucagaaugccacggugaauacguucccgggccuuguacacaccgcucgucacaccaugggaguggguuguaaaagaaguagguagcuuaacuucgggagggcgcuuaccacu

SEQ ID NO: 30 - RNA, 1541 nt, Streptococcus bovis
agaguuugauccuggcucaggacgaacgcuggcggcgugccuaauacaugcaaguagaacgcugaagacuuuagcuugcuaaaguuggaagaguugcgaacgggugaguaacgcguagguaaccugccuacuagcgggggauaacuauuggaaacgauagcuaauaccgcauaacagcauuuaacacauguuagaugcuugaaaggagcaauugcuucacuaguagauggaccugcguuguauuagcuaguuggugagguaacggcucaccaaggcgacgauacauagccgaccugagagggugaucggccacacugggacugagacacggcccagacuccuacgggaggcagcaguagggaaucuucggcaaugggggcaacccugaccgagcaacgccgcgugagugaagaagguuuucggaucguaaagcucuguuguaagagaagaacgugugugagaguggaaaguucacacagugacgguaacuuaccagaaagggacggcuaacuacgugccagcagccgcgguaauacguaggucccgagcguuguccggauuuauugggcguaaagcgagcgcaggcgguuuaauaagucugaaguuaaaggcaguggcuuaaccauuguucgcuuuggaaacuguuagacuugagugcagaaggggagaguggaauuccauguguagcggugaaaugcguagauauauggaggaacaccgguggcgaaagcggcucucuggucuguaacugacgcugaggcucgaaagcguggggagcaaacaggauuagauacccugguaguccacgccguaaacgaugagugcuagguguuaggcccuuuccggggcuuagugccgcagcuaacgcauuaagcacuccgccuggggaguacgaccgcaagguugaaacucaaaggaauugacgggggcccgcacaagcgguggagcaugugguuuaauucgaagcaacgcgaagaaccuuaccaggucuugacaucccgaugcuauuccuagagauaggaaguuucuucggaacaucggugacagguggugcaugguugucgucagcucgugucgugagauguuggguuaagucccgcaacgagcgcaaccccuauuguuaguugccaucauuaaguugggcacucuagcgagacugccgguaauaaaccggaggaagguggggaugacgucaaaucaucaugccccuuaugaccugggcuacacacgugcuacaaugguugguacaacgagucgcgagucggugacggcaagcaaaucucuuaaagccaaucucaguucggauuguaggcugcaacucgccuacaugaagucggaaucgcuaguaaucgcggaucagcacgccgcggugaauacguucccgggccuuguacacaccgcccgucacaccacgagaguuuguaacacccgaagucggugagguaaccuuuuaggagccagccgccuaaggugggauagaugauuggggugaagucguaacaagguagccguaucggaaggugcggcuggaucaccuccuu

SEQ ID NO: 31 - RNA, 1492 nt, Streptococcus infantarius
gcucaggacuaacgcuggcggcgugccuaauacaugcaaguagaacgcugaaaacuuuagcuugcuaaaguuugaagaguugcgaacgggugaguaacgcguagguaaccugccuacuagcgggggauaacuauuggaaacgauagcuaauaccgcauaacagcauuuaacccauguuagaugcuugaaaggagcaauugcuucacuaguagauggaccugcguuguauuagcuaguuggugagguaacggcucaccaaggcgacgauacauagccgaccugagagggugaucggccacacugggacugagacacggcccagacuccuacgggaggcagcaguagggaaucuucggcaaugggggcaacccugaccgagcaacgccgcgugagugaagaagguuuucggaucguaaagcucuguuguaagagaagaaugugugugagaguggaaaguucacacagugacgguaacuuaccagaaagggacggcuaacuacgugccagcagccgcgguaauacguaggucccgagcguuguccggauuuauugggcguaaagcgagcgcaggcgguuuaauaagucugaaguuaaaggcaguggcuuaaccauuguucgcuuuggaaacuguuagacuugagugcagaaggggagaguggaauuccauguguagcggugaaaugcguagauauauggaggaacaccgguggcgaaagcggcucucuggucuguaacugacgcugaggcucgaaagcguggggagcaaacaggauuagauacccugguaguccacgccguaaacgaugagugcuagguguuaggcccuuuccggggcuuagugccgcagcuaacgcauuaagcacuccgccuggggaguacgaccgcaagguugaaacucaaaggaauugacgggggcccgcacaagcgguggagcaugugguuuaauucgaagcaacgcgaagaaccuuaccaggucuugacaucccgaugcuauuccuagagauaggaaguuucuucggaacaucggugacagguggugcaugguugucgucagcucgugucgugagauguuggguuaagucccgcaacgagcgcaaccccuauuguuaguugccaucauuaaguugggcacucuagcgagacugccgguaauaaaccggaggaagguggggaugacgucaaaucaucaugccccuuaugaccugggcuacacacgugcuacaaugguugguacmacgagucgcgagucggugacggcaagcaaaucucuuaaagccaaucucaguucggauuguaggcugcaacucgccuacaugaagucggaaucgcuaguaaucgcggaucagcacgccgcggugaauacguucccgggccuuguacacaccgcccgucacaccacgagaguuuguaacacccgaagucggugagguaaccuuuuaggagccagccgccuaaggugggauagaugauuggggugaagucguaacaaggu

SEQ ID NO: 32 - RNA, 1487 nt, Streptococcus salivarius
  modified_base (939)..(939): a, c, g, u, unknown or other
  misc_feature (939)..(939): n is a, c, g, or u
  modified_base (1059)..(1059): a, c, g, u, unknown or other
  misc_feature (1059)..(1059): n is a, c, g, or u
uuuaaugagaguuugauccuggcucaggacgaacgcuggcggcgugccuaauacaugcaaguagaacgcugaagagaggagcuugcucuucuuggaugaguugcgaacgggugaguaacgcguagguaaccugccuuguagcgggggauaacuauuggaaacgauagcuaauaccgcauaacaauggaugacccaugucauuuauuugaaaggggcaaaugcuccacuacaagauggaccugcguuguauuagcuaguaggugagguaacggcucaccuaggcgacgauacauagccgaccugagagggugaucggccacacugggacugagacacggcccagacuccuacgggaggcagcaguagggaaucuucggcaaugggggcaacccugaccgagcaacgccgcgugagugaagaagguuuucggaucguaaagcucuguuguaagucaagaacgagugugagaguggaaaguucacacugugacgguagcuuaccagaaagggacggcuaacuacgugccagcagccgcgguaauacguaggucccgagcguuguccggauuuauugggcguaaagcgagcgcaggcgguuugauaagucugaaguuaaaggcuguggcucaaccauaguucgcuuuggaaacugucaaacuugagugcagaaggggagaguggaauuccauguguagcggugaaaugcguagauauauggaggaacaccgguggcgaaagcggcucucuggucuguaacugacgcugaggcucgaaagcguggggagcgaacaggauuagauacccugguaguccacgccguaaacgaugagugcuagguguuggauccuuuccgggauucagugccgcagcuaacgcauuaagcacuccgccuggggaguacgaccgcaagguugaaacucaaaggaauugacgggggccngcacaagcgguggagcaugugguuuaauucgaagcaacgcgaagaaccuuaccaggucuugacaucccgaugcuauuucuagagauagaaaguuacuucgguacaucggugacagguggngcaugguugucgucagcucgugucgugagauguuggguuaagucccgcaacgagcgcaaccccuauuguuaguugccaucauucaguugggcacucuagcgagacugccgguaauaaaccggaggaagguggggaugacgucaaaucaucaugccccuuaugaccugggcuacacacgugcuacaaugguugguacaacgaguugcgagucggugacggcaagcuaaucucuuaaagccaaucucaguucggauuguaggcugcaacucgccuacaugaagucggaaucgcuaguaaucgcggaucagcacgccgcggugaauacguucccgggccuuguacacaccgcccgucacaccacgagaguuuguaacacccgaagucggugagguaaccuuuuggagccagccgccuaaggugggauagau

SEQ ID NO: 33 - RNA, 1540 nt, Streptococcus thermophilus
  modified_base (130)..(130): a, c, g, u, unknown or other
  misc_feature (130)..(130): n is a, c, g, or u
agaguuugauccuggcucaggacgaacgguggcggcgugccuaauacaugcaaguagaacgcugaagagaggagcuugcucuucuuggaugaguugcgaacgggugaguaacgcguagguaaccugccunguagcgggggauaacuauuggaaacgauagcuaauaccgcauaacaauggaugacacaugucauuuauuugaaaggggcaauugcuccacuacaagauggaccugcguuguauuagcuaguaggugagguaauggcuuaccuaggcgacgauacauagccgaccugagagggugaucggccacacugggacugagacacggcccagacuccuacgggaggcagcaguagggaaucuucggcaaugggggcaacccugaccgagcaacgccgcgugacugaagaagguuuucggaucguaaagcucuguuguaagucaagaacgggugugagaguggaaaguucacacagugacgguagcuuaccagaaagggacggcuaacuacgugccagcagccgcgguaauacguaggucccgagcguuguccggauuuauugggcguaaagcgagcgcaggcgguuugauaagucugaaguuaaaggcuguggcucaaccauaguucgcuuuggaaacugucaaacuugagugcagaaggggagaguggaauuccauguguagcggugaaaugcguagauauauggaggaacaccgguggcgaaagcggcucucuggucuguaacugacgcugaggcucgaaagcguggggagcgaacaggauuagauacccugguaguccacgccguaaacgaugagugcuagguguuggauccuuuccgggauucagugccgcagcuaacgcauuaagcacuccgccuggggaguacgaccggaagguugaaacucaaaggaauugacggggcccgcacaagcgguggagcaugugguuuaauucgaagcaacgcgaagaaccuuaccaccucuugacaucccgaugcuauuucuagagauagaaaguuacuuugguacaucggugacagguggugcaugguugucgucagcucgugucgugagauguuggguuaagucccgcaacgagcgcaaccccuauuguuaguugccaucauucaguugggcacucuagcgagacugccgguaauaaaccggaggaagguggggaugacgucaaaucaucaugccccuuaugaccugggcuacacacgugcuacaaugguugguacaacgaguugcgagucggugacggcgagcuaaucucuuaaagccaaucucaguucggauuguaggcugcaacucgccuacaugaagucggaaucgcuaguaaucgcggaucagcacgccgcggugaauacguucccgggccuuguacacaccgcccgucacaccacgagaguuuguaacacccgaagucggugagguaaccuuuuggagccagccgccuaaggugggacagaugauuggggugaagucguaacaagguagccgauccggaaggugcggcuggaucaccuccuuu

SEQ ID NO: 34 - RNA, 1332 nt, Fusobacterium alocis
  modified_base (76)..(76): a, c, g, u, unknown or other
  misc_feature (76)..(76): n is a, c, g, or u
  modified_base (80)..(80): a, c, g, u, unknown or other
  misc_feature (80)..(80): n is a, c, g, or u
  modified_base (258)..(258): a, c, g, u, unknown or other
  misc_feature (258)..(258): n is a, c, g, or u
  modified_base (261)..(261): a, c, g, u, unknown or other
  misc_feature (261)..(261): n is a, c, g, or u
  modified_base (374)..(374): a, c, g, u, unknown or other
  misc_feature (374)..(374): n is a, c, g, or u
  modified_base (812)..(812): a, c, g, u, unknown or other
  misc_feature (812)..(812): n is a, c, g, or u
  modified_base (847)..(847): a, c, g, u, unknown or other
  misc_feature (847)..(847): n is a, c, g, or u
  modified_base (999)..(999): a, c, g, u, unknown or other
  misc_feature (999)..(999): n is a, c, g, or u
  modified_base (1076)..(1076): a, c, g, u, unknown or other
  misc_feature (1076)..(1076): n is a, c, g, or u
  modified_base (1174)..(1174): a, c, g, u, unknown or other
  misc_feature (1174)..(1174): n is a, c, g, or u
ggguggcggacgggugaguaacgcguaaagaacuugccucacagauagggacaacauuuagaaaugaaugcuaaunccunauauuaugaaaauagggcauccuauaauuaugaaagcuaaaugcgcugugagagagcuuugcgucccauuagcuaguuggagagguaacggcucaccaaggcgaugauggguagccggccugagagggugaacggccacaaggggacugagacacggcccuuacuccuacgggaggcngcnguggggaauauuggacaauggaccgagagucugauccagcaauucugugugcacgaugaaguuuuucggaauguaaagugcuuucaguugggaagaaaagaaugacgguaccnacagaagaagugacggcuaaauacgugccagcagccgcgguaauacguaugucacgagcguuauccggauuuauugggcguaaagcgcgucuaggugguuauguaagucugaugugaaaaugcagggcucaacucuguauugcguuggaaacuguguaacuagaguacuggagagguaagcggaacuacaaguguagaggugaaauucguagauauuuguaggaaugccgauggggaagccagcuuacuggacagauacugacgcuaaagcgcgaaagcguggguagcaaacaggauuagauacccugguaguccacgccguaaacgaugauuacuagguguugggggucgaaccucagcgcccaagcuaacgcgauaaguaauccgccuggggaguacguacgcaaguaugaaacucaaaggaauugacggggaccngcacaagcgguggagcaugugguuuaauucgacgnaacgcgaggaaccuuaccagcguuugacaucuuaggaaugagauagagauauaucagugucuucggggaaaccuaaagacagguggugcauggcugucgucagcucgugucgugagauguuggguuaagucccgcaacgagcgcaaccccunucguauguuaccaucauuaaguuggggacucaugcgauacugccugcgaugagcaggaggaagguggggaugacgunaagucaucaugccccuuauacgcugggcuacacacgugcuacaauggguagaacagagaguugcaaagccgugagguggagcuaaucucagaaaacunuucuuaguucggauuguacucugcaacucgaguacaugaaguuggaaucgcuaguaaucgcgaaucagcaaugucgcggugaauacguucucgggucuuguacacaccgcccgucacaccacgagaguugguugcaccugaaguagcaggccuaaccg

SEQ ID NO: 35 - RNA, 1461 nt, Fusobacterium nucleatum
  modified_base (288)..(288): a, c, g, u, unknown or other
  misc_feature (288)..(288): n is a, c, g, or u
  modified_base (637)..(637): a, c, g, u, unknown or other
  misc_feature (637)..(637): n is a, c, g, or u
  modified_base (744)..(746): a, c, g, u, unknown or other
  misc_feature (744)..(746): n is a, c, g, or u
  modified_base (941)..(941): a, c, g, u, unknown or other
  misc_feature (941)..(941): n is a, c, g, or u
auugaacgaagaguuugauccuggcucaggaugaacgcugacagaaugcuuaacacaugcaagucuacuugaauuuggguuuuuuaacuucgauuuggguggcggacgggugaguaacgcguaaagaacuugccucacagcuagggacaacauuuggaaacgaaugcuaauaccuaauauuaugauuauagggcauccuagaauuaugaaagcuauaugcgcugugagagagcuuugcgucccauuagcuaguuggagagguaacggcucaccaaggcaaugaugggnagccggccugagagggugaacggccacaaggggacugagacacggcccuuacuccuacgggaggcagcaguggggaauauuggacaauggaccgagagucugauccagcaauucugugugcacgaugacguuuuucggaauguaaagugcuuucaguugggaagaaaaaaaugacgguaccaacagaagaagugacggcuaaauacgugccagcagccgcgguaauacguaugucacgagcguuauccggauuuauugggcguaaagcgcgucuaggugguuauguaagucugaugugaaaaugcagggcucaacucuguauugcguuggaaacuguguaacuagaguncuggaaagguaagcggaacuacaaguguagaggugaaauucguagauauuuguaggaaugccgauggggaagccagcuuacuggacagauacugacgcugaagcgcnnnagcguggguagcaaacaggauuagauacccugguaguccacgccguaaacgaugauuacuagguguugggggucgaaccucagcgcccaagcaaacgcgauaaguaauccgccuggggaguacguacgcaaguaugaaacucaaaggaauugacggggacccgcacaagcgguggagcaugugguuuaauucgangcaacgcgaggaaccuuaccagcguuugacaucuuaggaaugagacagagauguuucagugucccuucggggaaaccuaaagacagguggugcauggcugucgucagcucgugucgugagauguuggguuaagucccgcaacgagcgcaaccccuuucguauguuaccaucauuaaguuggggacucaugcgauacugccuacgaugaguaggaggaagguggggaugacgucaagucaucaugccccuuauacgcugggcuacacacgugcuacaauggguagaacagagaguugcaaagccgugagguggagcuaaucucagaaaacuauucuuaguucggauuguacucugcaacucgaguacaugaaguuggaaucgcuaguaaucgcgaaucagcaaugucgcggugaauacguucucgggucuuguacacaccgcccgucacaccacgagaguugguugcaccugaaguagcaggccuaaccguaaggagggauguuccgagggugugauuagc

SEQ ID NO: 36 - RNA, 1452 nt, Fusobacterium varium
  modified_base (256)..(256): a, c, g, u, unknown or other
  misc_feature (256)..(256): n is a, c, g, or u
  modified_base (271)..(271): a, c, g, u, unknown or other
  misc_feature (271)..(271): n is a, c, g, or u
  modified_base (320)..(320): a, c, g, u, unknown or other
  misc_feature (320)..(320): n is a, c, g, or u
  modified_base (422)..(422): a, c, g, u, unknown or other
  misc_feature (422)..(422): n is a, c, g, or u
  modified_base (509)..(509): a, c, g, u, unknown or other
  misc_feature (509)..(509): n is a, c, g, or u
  modified_base (683)..(683): a, c, g, u, unknown or other
  misc_feature (683)..(683): n is a, c, g, or u
  modified_base (844)..(844): a, c, g, u, unknown or other
  misc_feature (844)..(844): n is a, c, g, or u
  modified_base (891)..(891): a, c, g, u, unknown or other
  misc_feature (891)..(891): n is a, c, g, or u
  modified_base (923)..(926): a, c, g, u, unknown or other
  misc_feature (923)..(926): n is a, c, g, or u
  modified_base (998)..(998): a, c, g, u, unknown or other
  misc_feature (998)..(998): n is a, c, g, or u
  modified_base (1157)..(1158): a, c, g, u, unknown or other
  misc_feature (1157)..(1158): n is a, c, g, or u
  modified_base (1447)..(1447): a, c, g, u, unknown or other
  misc_feature (1447)..(1447): n is a, c, g, or u
aacgaagaguuugauccuggcucaggaugaacgcugacagaaugcuuaacacaugcaagucuacuugaucuucgggugaagguggcggacgggugaguaacgcguaaagaacuugccuuacagacugggacaacauuuggaaacgaaugcuaauaccggauauuaugacugagucgcaugauuugguuaugaaagcuauaugcgcugugagagagcuuugcgucccauuaguuaguuggugagguaacggcucacnaagacgaugaugggnagccggccugagagggugaacggccacaaggggacugagacacggcccnyacuccuacgggaggcagcaguggggaauauuggacaauggaccaaaagucugauccagcaauucugugugcacgaugaaguuuuucggaauguaaagugcnuucaguugggaagaagucagugacgguaccaacagaagaagcgacggcuaaauacgugccagcagccgcgguaauacguaugucgcnagcguuauccggauuuauugggcguaaagcgcgucuaggcgguuuaguaagucugaugugaaaaugcggggcucaaccccguauugcguuggaaacugcuaaacuagaguacuggagagguaggcggaacuacaaguguagaggugaaauucguagauauuuguaggaaugccnauggggaagccagccuacuggacagauacugacgcuaaagcgcgaaagcguggguagcaaacaggauuagauacccugguaguccacgccguaaacgaugauuacuagguguugggggucgaaccucagcgcccaagcuaacgcgauaaguaauccgccungggaguacguacgcaaguaugaaacucaaaggaauugacggggaccngcacaagcgguggagcaugugguuuaauucgnnnnaacgcgaggaaccuuaccagcguuugacaucccaagaaguuaacagagauguuuucgugccucuucggaggnacuuggugacagguggugcauggcugucgucagcucgugucgugagauguuggguuaagucccgcaacgagcgcaaccccuuucguauguuaccaucauuaaguuggggacucaugcgagacugccugcgaugagcaggaggaagguggggaugacgunnagucaucaugccccuuauacgcugggcuacacacgugcuacaauggguaguacagagagcugcaaaccugcgaggguaagcuaaucucauaaaacuauucuuaguucggauuguacucugcaacucgaguacaugaaguuggaaucgcuaguaaucgcaaaucagcuauguugcggugaauacguucucgggucuuguacacaccgcccgucacaccacgagaguugguugcaccugaaguaacaggccuaaccguaaggagggauguuccgagggugugauuagcganugggg

SEQ ID NO: 37 - DNA, 20 bp, Artificial Sequence
  Synthetic oligonucleotide
agagtttgatcctggctcag

SEQ ID NO: 38 - DNA, 18 bp, Artificial Sequence
  Synthetic oligonucleotide
gctgcctcccgtaggagt